(19) United States
(12) Patent Application Publication
(10) Pub. No.: US 2005/0244367 A1
(43) Pub. Date: Nov. 3, 2005

(54) Phospholipase inhibitors localized in the gastrointestinal lumen

(75) Inventors: David Hui, Cincinnati, CA (US); Dominique Charmot, Campbell, CA (US); Jerry Buysse, Los Altos, CA (US)

Correspondence Address: WILSON SONSINI GOODRICH & ROSATI, 650 PAGE MILL ROAD, PALO ALTO, CA, 94304-1050, US

(73) Assignee: Ilypsa, Inc., Santa Clara, CA (US)

(21) Appl. No.: 10/838,879

(22) Filed: May 3, 2004

(51) Int. Cl. A61K 31/74; A61K 31/785; A61K 31/66

(52) U.S. Cl. 424/78.27; 514/102; 514/129

(57) ABSTRACT

The present invention provides methods and compositions for the treatment of phospholipase-related conditions. In particular, the invention provides a method of treating insulin-related, weight-related conditions and/or cholesterol-related conditions in an animal subject. The method generally involves the administration of a non-absorbed and/or effluxed phospholipase A2 inhibitor that is localized in a gastrointestinal lumen.

### BACKGROUND OF THE INVENTION

Phospholipases are a group of enzymes that play important roles in a number of biochemical processes, including regulation of membrane fluidity and stability, digestion and metabolism of phospholipids, and production of intracellular messengers involved in inflammatory pathways, hemodynamic regulation and other cellular processes. Phospholipases are themselves regulated by a number of mechanisms, including selective phosphorylation, pH, and intracellular calcium levels. Phospholipase activities can be modulated to regulate their related biochemical processes, and a number of phospholipase inhibitors have been developed.

Certain phospholipase activities occur in the gastrointestinal lumen, for example, phospholipase A2 acts in the digestion of dietary phospholipids in the gastrointestinal lumen, and phospholipase B is active in the apical mucosa of the distal intestine. The activities of these enzymes affect a number of phospholipase-related conditions, including diabetes, weight gain and cholesterol-related conditions.

Diabetes affects 18.2 million people in the Unites States, representing over 6% of the population. Diabetes is characterized by the inability to produce or properly use insulin. Diabetes type 2 (also called non-insulin-dependent diabetes or NIDDM) accounts for 80-90% of the diagnosed cases of diabetes and is caused by insulin resistance. Insulin resistance in diabetes type 2 prevents maintenance of blood glucose within desirable ranges, despite normal to elevated plasma levels of insulin.

Obesity is a major contributor to diabetes type 2, as well as other illnesses including coronary heart disease, osteoarthritis, respiratory problems, and certain cancers. Despite attempts to control weight gain, obesity remains a serious health concern in the United States and other industrialized countries. Indeed, over 60% of adults in the United States are considered overweight, with about 22% of these being classified as obese.

Diet also contributes to elevated plasma levels of cholesterol, including non-HDL cholesterol. Non-HDL cholesterol is associated with atherogenesis and its sequalea including arteriosclerosis, myocardial infarction, ischemic stroke, and other forms of heart disease that together rank as the most prevalent type of illness in industrialized countries. Indeed, an estimated 12 million people in the United States suffer with coronary artery disease and about 36 million require treatment for elevated cholesterol levels.

With the high prevalence of diabetes, obesity, and cholesterol-related conditions, there remains a need for approaches that treat one or more of these conditions, including reducing unwanted side effects. The present invention provides methods, compositions, and kits for using phospholipase inhibitors to treat phospholipase-related conditions, such as insulin-related conditions (e.g., diabetes), weight-related conditions (e.g., obesity) and/or cholesterol-related conditions.

### BRIEF SUMMARY OF THE INVENTION

One aspect of the present invention relates to a composition comprising a phospholipase inhibitor wherein said inhibitor is localized in a gastrointestinal lumen. In some embodiments, the inhibitor is not absorbed through a gastrointestinal mucosa. In some embodiments, the inhibitor is designed to have low permeability; in some embodiments, the inhibitor comprises a phospholipase inhibiting moiety linked to a non-absorbed moiety, preferably a polymer moiety. In some embodiments, the phospholipase inhibiting moiety is a phospholipid analog or a transition state analog that is linked via its hydrophobic group. In some embodiments, the inhibitor is localized in the gastrointestinal lumen as a result of efflux from a gastrointestinal mucosal cell.

In some embodiments, the inhibitor acts by hindering access of a phospholipase to its phospholipid substrate. In some embodiments, the inhibitor interacts with a lipid-water interface of a lipid aggregate containing phospholipid substrate; in some embodiments the inhibitor interacts with the phospholipase, preferably with the catalytic site bearing face of PLA2. In some embodiments, the inhibitor acts by interacting with a specific site on a phospholipase, e.g., the catalytic site, reversibly or irreversibly.

In some embodiments, the inhibitor inhibits phospholipase A2; in some embodiments, the inhibitor inhibits phospholipase A2 and phospholipase B. In some embodiments, the inhibitor inhibits phospholipase A2 but essentially does not inhibit phospholipase B; in some embodiments the inhibitor essentially does not inhibit a lipase. In some embodiments, the inhibitor does not act on the gastrointestinal mucosa. In some embodiments, the phospholipase inhibitors herein can produce a therapeutic and/or a prophylactic benefit in treating an insulin-related condition (e.g., diabetes type 2), a weight-related condition (e.g., obesity), and/or a cholesterol-related condition (e.g., hypercholesterolemia) in a subject receiving said inhibitor.

Another aspect of the invention provides methods of inhibiting a phospholipase by administering an effective amount of a composition herein to a subject in need thereof. Preferred embodiments provide a method of treating a condition by administering an effective amount of a phospholipase inhibitor to a subject in need thereof where the inhibitor is not absorbed through a gastrointestinal mucosa and/or where the inhibitor is localized in a gastrointestinal lumen as a result of efflux from a gastrointestinal mucosal cell. Preferably, the condition treated is an insulin-related condition (e.g., diabetes type 2), a weight-related condition (e.g., obesity) and/or a cholesterol-related condition (e.g., hypercholesterolemia).

Yet another aspect of the invention relates to a method of making a phospholipase inhibitor that is localized in a gastrointestinal lumen by contacting a candidate moiety with a phospholipase A2, a lipid-water interface, phospholipase B, or fragment thereof; determining whether the candidate moiety interacts with the phospholipase A2, interface, phospholipase B, or fragment thereof; selecting said candidate moiety that interacts with phospholipase A2, interface, phospholipase B, or fragment thereof; and using the selected candidate moiety as a phospholipase A2 or phospholipase B inhibiting moiety of the phospholipase inhibitor that is localized in the gastrointestinal lumen. In some embodiment, a candidate moiety is selected that does not interact with phospholipase B or fragment thereof.

### BRIEF DESCRIPTION OF THE DRAWINGS

FIG. 1(a) shows interaction of a phospholipase with a lipid-water interface; FIG. 1(b) illustrates a non-absorbed phospholipase inhibitor that interacts with a lipid-water interface; FIG. 1(c) illustrates a non-absorbed phospholipase inhibitor that interacts with the enzyme.

### DETAILED DESCRIPTION OF THE INVENTION

One aspect of the present invention provides phospholipase inhibitors. The phospholipase inhibitors act in the gastrointestinal lumen, preferably to modulate the absorption and/or downstream activities of products of phospholipase digestion. The phospholipase inhibitors of the present invention find use in treating a number of phospholipase-related conditions, including insulin-related conditions (e.g., diabetes), weight-related conditions (e.g., obesity), cholesterol-related disorders and any combination thereof, as described in detail below.

In some embodiments, phospholipase inhibitors of the present invention blunt or reduce the catalytic activity of phospholipases, preferably phospholipases secreted or localized in the gastrointestinal tract, including the gastric compartment, and more particularly the small intestine. For example, such enzymes include, but are not limited to, secreted Group IB phospholipase A2 (PLA2-IB), also referred to as pancreatic phospholipase A2 (p-PLA2) and herein referred to as “PLA2” or “phospholipase A2;” secreted Group IIA phospholipase A2 (PLA2 IIA); phospholipase A1 (PLA1); phospholipase B (PLB); phospholipase C (PLC); and phospholipase D (PLD). In some preferred embodiments, inhibitors of the present invention do not inhibit or do not significantly inhibit or essentially do not inhibit lipases, such as pancreatic triglyceride lipase (PTL) and carboxyl ester lipase (CEL).

In some preferred embodiments, inhibitors of the present invention inhibit PLA2, but do not inhibit or do not significantly inhibit or essentially do not inhibit any other phospholipases; in some preferred embodiments, inhibitors of the present invention inhibit PLA2 but do not inhibit or do not significantly inhibit or essentially do not inhibit PLA1; in some preferred embodiments, inhibitors of the present invention inhibit PLA2 but do not inhibit or do not significantly inhibit or essentially do not inhibit PLB. In some embodiments, the phospholipase inhibitor does not act on the gastrointestinal mucosa, for example, it does not inhibit or does not significantly inhibit or essentially does not inhibit membrane-bound phospholipases. The different activities of PLA2, PLA1, and PLB are well-known in the art. PLA2 hydrolyzes phospholipids at the sn-2 position liberating 1-acyl lysophospholipids and fatty acids; PLA1 acts on phospholipids at the sn-1 position to release 2-acyl lysophospholipids and fatty acids; while phospholipase B cleaves phospholipids at both sn-1 and sn-2 positions to form a glycerol and two fatty acids. See, e.g., Devlin, Editor, Textbook of Biochemistry with Clinical Correlations, 5^{th} ed. Pp 1104-1110 (2002). Phospholipids acted upon by gastrointestinal PLA1, PLA2 and PLB are mostly of the phosphatidycholine and phosphatidylethanolamine types, and can be of dietary or biliary origin, or may be derived from being sloughed off of cell membranes. For example, in the case of phosphatidylcholine digestion, PLA1 acts at the sn-1 position to produce 2-acyl lysophosphatidylcholine and free fatty acid; PLA2 acts at the sn-2 position to produce 1-acyl lysophosphatidylcholine and free fatty acid; while PLB acts at both positions to produce glycerol 3-phosphorylcholine and two free fatty acids (Devlin, 2002).

As is also known in the art, pancreatic PLA2 is secreted by acinar cells of the exocrine pancreas for release in the duodenum via pancreatic juice. PLA2 is secreted as a proenzyme, carrying a polypeptide chain that is subsequently cleaved by proteases to activate the enzyme's catalytic site. Documented structure-activity-relationships (SAR) for PLA2 isozymes illustrate a number of common features (see for instance, Gelb M., Chemical Reviews, 2001, 101:2613-2653; Homan, R., Advances in Pharmacology, 1995, 12:31-66; and Jain, M. K., Intestinal Lipid Metabolism, Biology, pathology, and interfacial enzymology of pancreatic phospholipase A2, 2001, 81-104, each incorporated herein by reference). The inhibitors of the present invention can take advantage of certain of these common features to inhibit PLA2 activity. Common features of PLA2 enzymes include sizes of about 13 to about 15 kDa; stability to heat; and 6 to 8 disulfides bridges. Common features of PLA2 enzymes also include conserved active site architecture and calcium-dependent activities, as well as a catalytic mechanism involving concerted binding of His and Asp residues to water molecules and a calcium cation, in a His-calcium-Asp triad. A phospholipid substrate can access the catalytic site by its polar head group through a slot enveloped by hydrophobic and cationic residues (including lysine and arginine residues) described in more detail below. Within the catalytic site, the multi-coordinated calcium ion activates the acyl carbonyl group of the sn-2 position of the phospholipid substrate to bring about hydrolysis (Devlin, 2002). In some preferred embodiments, inhibitors of the present invention inhibit this catalytic activity of PLA2 by interacting with its catalytic site.

It is also known that most PLA2 enzymes act almost exclusively at the lipid-water interface of lipid aggregates found in the gastrointestinal lumen, including, for example, fat globules, emulsion droplets, vesicles, mixed micelles, and/or disks, any one of which may contain triglycerides, fatty acids, bile acids, phospholipids, phosphatidylcholine, lysophospholipids, lysophosphatidylcholine, cholesterol, cholesterol esters, other amphiphiles and/or other diet metabolites. Such enzymes act while “docked” to a lipid-water interface. In such lipid aggregates, the phospholipid substrates are arranged in a mono or bilayer, together with one or more other components listed above, which form part of the outer surface of the aggregate. The surface of a phospholipase bearing the catalytic site contacts this interface facilitating access to phospholipid substrates. This surface of the phospholipase is known as the i-face, i.e., the interfacial recognition face of the enzyme. The structural features of the i-face of PLA2 have been well documented. See, e.g., Jain, M. K, et al, Methods in Enzymology, vol. 239, 1995, 568-614, incorporated herein by reference. The inhibitors of the present invention can take advantage of these structural features to inhibit PLA2 activity. For instance, it is known that the aperture of the slot forming the catalytic site is normal to the i-face plane. The aperture is surrounded by a first crown of hydrophobic residues (mainly leucine and isoleucine residues), which itself is contained in a ring of cationic residues (including lysine and arginine residues). In some preferred embodiments, inhibitors of the present invention hinder access of PLA2 to its phospholipid substrates by interacting with this i-face and/or with the lipid-water interface.

Moreover, the localized action of phospholipases, e.g. PLA2, in digesting phospholipid substrates suggests a localized approach, pursuant to the present invention. That is, the present invention provides phospholipase inhibitors that are not absorbed through a gastrointestinal mucosa and/or are effluxed back into a gastrointestinal lumen, as described in detail below. Such inhibitors can be used in the treatment of phospholipase-related conditions, preferably phospholipase A2-related conditions and phospholipase A2-related conditions induced by diet, including but not limited to diabetes (e.g. diabetes type 2 and other insulin-related conditions), weight-related conditions (e.g., obesity), cholesterol-related conditions and combinations thereof.

In some embodiments, the phospholipase inhibitors of the present invention are localized in a gastrointestinal lumen and are also cell impermeable, e.g., not internalized into a cell. In some embodiments, the phospholipase inhibitors are cell permeable, e.g., can be internalized into a cell, and are also localized in a gastrointestinal lumen. In these embodiments, gastrointestinal localization can be facilitated by an efflux mechanism, such as those further described below.

In some embodiments, the inhibitor is localized in the gastrointestinal lumen of an animal subject. The term “gastrointestinal lumen” is used interchangeably herein with the term “lumen,” to refer to the space or cavity within a gastrointestinal tract, which can also be referred to as the gut of the animal. In some embodiments, the phospholipase inhibitor is not absorbed through a gastrointestinal mucosa. “Gastrointestinal mucosa” refers to the layer(s) of cells separating the gastrointestinal lumen from the rest of the body and includes gastric and intestinal mucosa, such as the mucosa of the small intestine. In some embodiments, lumen localization is achieved by efflux into the gastrointestinal lumen upon uptake of the inhibitor by a gastrointestinal mucosal cell. A “gastrointestinal mucosal cell” as used herein refers to any cell of the gastrointestinal mucosa, including, for example, an epithelial cell of the gut, such as an intestinal enterocyte, a colonic enterocyte, an apical enterocyte, and the like. Such efflux achieves a net effect of non-absorbedness, as the terms, related terms and grammatical variations, are used herein.

“Not absorbed” as used herein refers to situations in which a significant amount, preferably a statistically significant amount, more preferably essentially all of the phospholipase inhibitor, remains in the gastrointestinal lumen. For example, at least about 90% of phospholipase inhibitor remains in the gastrointestinal lumen, at least about 95%, at least about 98%, preferably at least about 99%, and more preferably at least about 99.5% remains in the gastrointestinal lumen. In such cases, localization to the gastrointestinal lumen refers to reducing net movement across a gastrointestinal mucosa, for example, by way of both transcellular and paracellular transport, as well as by active and/or passive transport. The phospholipase inhibitor in such embodiments is hindered from net permeation of a gastrointestinal mucosal cell in transcellular transport, for example, through an apical cell of the small intestine; the phospholipase inhibitor in these embodiments is also hindered from net permeation through the “tight junctions” in paracellular transport between gastrointestinal mucosal cells lining the lumen. The term “not absorbed” is used interchangeably herein with the terms “non-absorbed,” “non-absorbedness,” “non-absorption” and its other grammatical variations.

In some embodiments, non-absorbedness is based on the charge, size, and/or other physical parameters of the phospholipase inhibitor. For example, in some embodiments, the phospholipase inhibitor is constructed to have a molecular structure that minimizes or nullifies absorption through a gastrointestinal mucosa. The absorption character of a drug can be selected by applying principles of pharmacodynamics, for example, by applying Lipinsky's rule, also known as “the rule of five.” Although not a rule, but rather a set of guidelines, Lipinsky shows that, small molecule drugs with (i) molecular weight, (ii) number of hydrogen bond donors, (iii) number of hydrogen bond acceptors, and (iv) water/octanol partition coefficient (Moriguchi logP) each greater than a certain threshold value generally do not show significant systemic concentration. See Lipinsky et al, Advanced Drug Delivery Reviews, 46, 2001 3-26, incorporated herein by reference. Accordingly, non-absorbed phospholipase inhibitors can be constructed to have molecule structures exceeding one or more of Lipinsky's threshold values. See also Lipinski et al., Experimental and computational approaches to estimate solubility and permeability in drug discovery and development settings, Adv. Drug Delivery Reviews, 46:3-26 (2001); and Lipinski, Drug-like properties and the causes o poor solubility and poor permeability, J. Pharm. & Toxicol. Methods, 44:235-249 (2000), incorporated herein by reference. In some preferred embodiments, for example, a phospholipase inhibitor of the present invention can be constructed to feature one or more of the following characteristics: (i) having a MW greater than about 500 Da; (ii) having a total number of NH and/or OH and/or other potential hydrogen bond donors greater than about 5; (iii) having a total number of O atoms and/or N atoms and/or other potential hydrogen bond acceptors greater than about 10; and/or (iv) having a Moriguchi partition coefficient greater than about 10⁵, i.e., logP greater than about 5. Any art known phospholipase inhibitors and/or any phospholipase inhibiting moieties described below can be used in constructing a non-absorbed molecular structure.

Because there are many exceptions to Lipinski's “rule,” preferably, the permeability properties of the compounds are screened experimentally: permeability coefficient can be determined by methods known to those of skill in the art, including for example by Caco-2 cell permeability assay and/or using an artificial membrane as a model of a gastrointestinal mucosa. For example, a synthetic membrane can be impregnated with e.g. lecithin and/or dodecane to mimic the net permeability characteristics of a gastrointestinal mucosa. The membrane can be used to separate a compartment containing the phospholipase inhibitor from a compartment where the rate of permeation will be monitored. Also, parallel artificial membrane permeability assays (PAMPA) can be performed. Such in vitro measurements can reasonably indicate actual permeability in vivo. See, for example, Wohnsland et aL J. Med. Chem., 2001, 44:923-930; Schmidt et al., Millipore corp. Application note, 2002, n° AN1725EN00, and n° AN1728EN00, incorporated herein by reference. The permeability coefficient is reported as its decimal logarithm, Log Pe.

In some embodiments, the phospholipase inhibitor permeability coefficient Log Pe is preferably lower than about −4, or lower than about −4.5, or lower than about −5, more preferably lower than about −5.5, and even more preferably lower than about −6 when measured in the permeability experiment described in Wohnsland et al. J. Med. Chem. 2001, 44. 923-930.

In some embodiments, a phospholipase inhibitor is constructed as described above to hinder its (net) absorption through a gastrointestinal mucosa. In some embodiments, a phospholipase inhibitor comprises a phospholipase inhibiting moiety linked, coupled or otherwise attached to a non-absorbed oligomer moiety, polymer moiety, hydrophobic moiety, hydrophilic moiety, and/or charged moiety. In some preferred embodiments, the phospholipase inhibiting moiety is coupled to a polymer moiety. The polymer moiety may be of molecular weight range from about 1000 Da to about 500,000 Da, preferably in the range of about 5000 to about 200,000 Da, and more preferably sufficiently high to hinder or preclude (net) absorption through a gastrointestinal mucosa. For example, a phospholipase inhibiting moiety may be linked to at least one repeat unit of a polymer moiety according to the following formula:

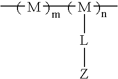

where n and m are each intergers, M represents a polymer moiety, L is a linking moiety, e.g., a chemical linker, and Z is a phospholipase inhibiting moiety, preferably a PLA2 inhibiting moiety.

The linking moiety L can be a chemical linker, such as a bond or a other moiety, for example, comprising about 1 to about 10 atoms that can be hydrophilic and/or hydrophobic. The linking moiety links, couples, or otherwise attaches the phospholipase inhibiting moiety Z to the polymer moiety, for example to a backbone of the polymer moiety.

The number of phospholipase inhibiting moieties Z appended to the polymer moiety can vary from about 1 to about 2000, most preferably from about I to about 500. These phospholipase inhibiting moieties can be arranged regularly or randomly along a backbone of the polymer moiety or can be localized in one particular region of the polymer moiety. For instance, (M) and (M-L-Z) repeat units can be arranged regularly, e.g., in sequences, or randomly along a backbone of the polymer moiety. If block copolymers are used, the phospholipase inhibiting moieties can be present on one block while not on another block.

The phospholipase inhibiting moiety Z may be any art-known phospholipase inhibitor, and/or any phospholipase inhibiting moiety described herein. Preferably, the phospholipase inhibitor comprises a phospholipase inhibiting moiety that is active under the physiological conditions of the GI tract, e.g. within the pH range prevailing within the gastrointestinal lumen, i.e., from about 5 to about 8, and preferably under physiological conditions prevailing at a location within the GI tract where the phospholipase inhibiting moiety acts, e.g., within the gastrointestinal lumen of the small intestine.

In some embodiments, non-absorbed PLA2 inhibitors of the invention comprise an art-know PLA2 inhibiting moiety. Art-know PLA2 inhibiting moieties include, for example, small molecule inhibitors of phospholipase A2, such as FPL 67047XX and/or MJ99. Other phospholipase inhibitors useful in the practice of the methods of this invention include arachidonic acid analogues (e.g., arachidonyl trifluoromethyl ketone, methylarachidonyl fluorophosphonate, and palmitoyl trifluoromethyl ketone), benzensulfonamide derivatives, bromoenol lactone, p-bromophenyl bromide, bromophenacyl bromide, trifluoromethylketone, sialoglycolipids, proteoglycans, and the like, as well as phospholipase A2 inhibitors disclosed in WO 03/101487, incorporated herein by reference.

Art-know PLA2 inhibiting moieties useful in this invention also include, for example, phospholipid analogs and structures developed to target secreted PLA2, for example, for indications such as obstructive respiratory disease (including asthma), colitis, Crohn's disease, central nervous system insult, ischemic stroke, multiple sclerosis, contact dermatitis, psoriasis, cardiovascular disease (including arteriosclerosis), autoimmune disease, and other inflammatory states.

Phospholipid analogs useful as phospholipase inhibiting moieties of some phospholipase inhibitors of this invention include structural analogs of a phospholipid substrate and/or its transition state, which can comprise one or more classes of compounds known in the art to resemble phospholipid substrates and/or their transition states, preferably resembling their polar head groups rather than their long chain hydrophobic groups. Such analog inhibitors can include, for example, compounds disclosed in Gelb M., Jain M., Berg O., Progress in Surgery, Principles of inhibition of phospholipase A2 and other interfacial enzymes, 1997, 24:123-129, for example, see Table 1 therein, incorporated herein by reference. Examples of PLA2 inhibiting moieties in some preferred embodiments are provided below:

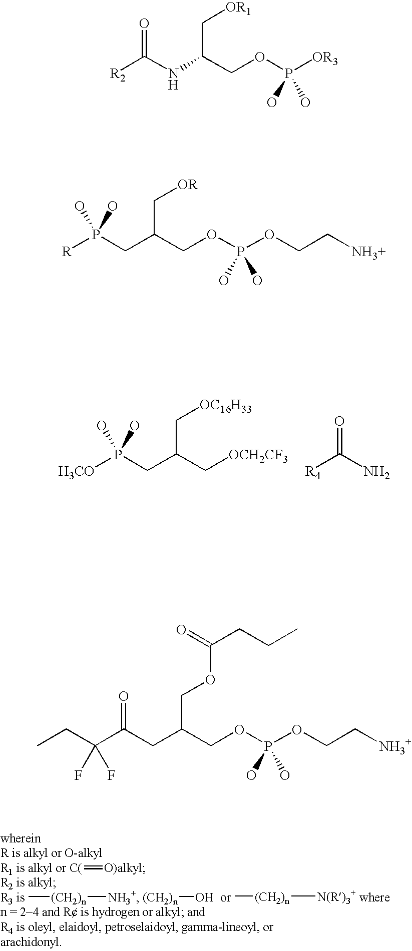

Phospholipid analogs useful as phospholipase inhibiting moieties of some phospholipase inhibitors of this invention also include phosphonate-containing compounds, such as those disclosed in Lin et al, J. Am. Chem. Soc., 115 (10) 1993, preferably the compounds represented by the structures provided below:

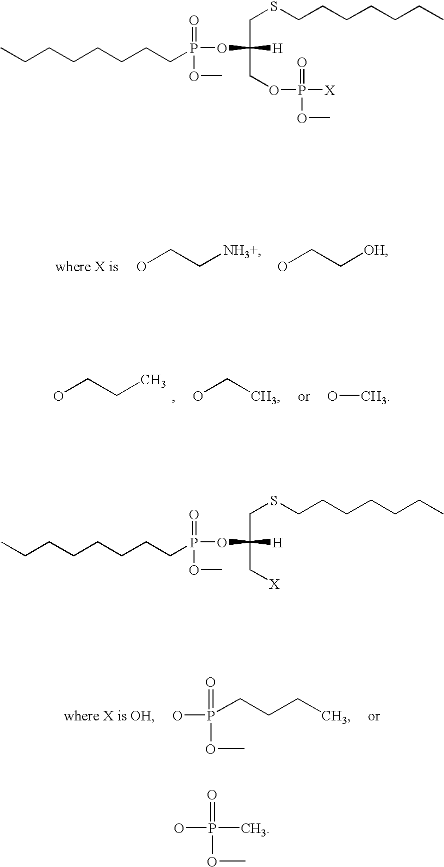

Transition state analogs useful as phospholipase inhibiting moieties of some phospholipid inhibitors of the present invention include one or more compounds taught in Jain, M et al., Biochemistry, 1991, 30:10256-10268, for example, see Tables IV, V and VI therein, incorporated herein by reference. In some preferred embodiments, inhibitors of the present invention comprise a moiety derived from modified glycerol backbone (see, for example, table VI of Jain, 1991), which have proven to be potent inhibitors of pancreatic PLA2, including, for example, the structures illustrated below:

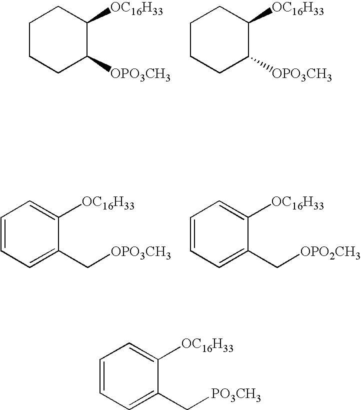

Certain indole glyoxamides are particularly useful as PLA2 inhibiting moieties in some embodiments, specifically Me-Indoxam represented by the structure below:

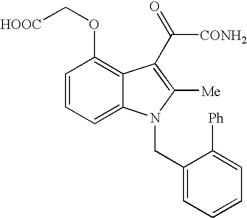

Other art-know phospholipase A2 inhibitors useful as phospholipase inhibiting moieties of the present invention include the following classes: Alkynoylbenzoic, -Thiophenecarboxylic, -Furancarboxylic, and -Pyridinecarboxylic acids (e.g. see U.S. Pat. No. 5,086,067); Amide carboxylate derivatives (e.g. see WO9108737); Aminoacid esters and amide derivatives (e.g. see WO2002008189); Aminotetrazoles (e.g. see U.S. Pat. No. 5,968,963); Aryoxyacle thiazoles (e.g. see WO00034254); Azetidinones (e.g. see WO9702242); Benzenesulfonic acid derivatives (e.g. see U.S. Pat. No. 5,470,882); Benzoic acid derivatives (e.g. see JP08325154); Benzothiaphenes (e.g. see WO 02000641); Benzyl alcohols (e.g. see U.S. Pat. No. 5,124,334); Benzyl phenyl pyrimidines (e.g. see WO00027824); Benzylamines (e.g. see U.S. Pat. No. 5,039,706); Cinammic acid compounds (e.g. see JP07252187); Cinnamic acid derivatives (e.g. see U.S. 5,578,639); Cyclohepta-indoles (e.g. see WO03016277); Ethaneamine-benzenes; Imidazolidinones, Thiazoldinones and Pyrrolidinones (e.g. see WO03031414); Indole glyoxamides (e.g. see U.S. Pat. No. 5,654,326); Indole glyoxamides (e.g. see WO9956752); Indoles (e.g. see U.S. Pat. No. 6,630,496 and WO9943672; Indoly (e.g. see WO003048122); Indoly containing sulfonamides; N-cyl-N-cinnamoylethylenedianine derivatives (e.g. see WO9603371); Naphyl acateamides (e.g. see EP77927); N-substituted glycines (e.g. see U.S. 5,298,652); Phosopholipid analogs (e.g. see U.S. Pat. No. 5,144,045 and U.S. Pat. No. 6,495,596); Piperazines (e.g. see WO03048139); Pyridones and Pyrimidones (e.g. see WO03086400); 6-carbamoylpicolinic acid derivatives (e.g. see JP07224038); Steroids and their cyclic hydrocarbon analogs with amino-containing sidechains (e.g. see WO8702367); Trifluorobutanones (e.g. see U.S. Pat. No. 6,350,892 and US2002068722); Abietic derivatives (e.g. see U.S. Pat. No. 4,948,813); Benzyl phosphinate esters (e.g. see U.S. Pat. No. 5,504,073); each of which is incorporated herein by reference.

Specific examples of phospholipase inhibiting moieties of some of these PLA2 inhibitor classes are provided in Table 1 below, along with IC50 values corresponding thereto:

| Example of phospholipase inhibiting moiety from a PLA2 inhibitor class | IC50 |
|---|---|
| 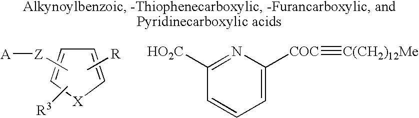 | μM range |
| 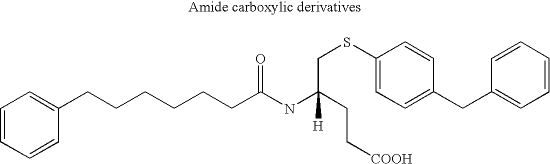 | sub μM range |
| Aminoacid esters and amide derivatives | about 2.5 μM |
| 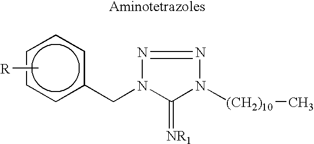 | μM range |
| 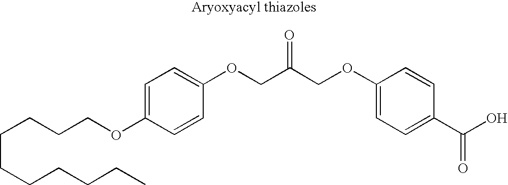 | |
| 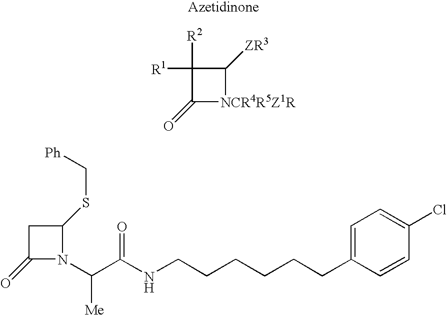 | |
| 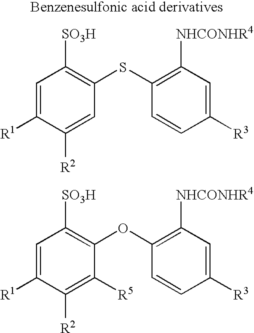 | |
| Benzoic acid derivatives | μM range |
| Benzothiaphenes | about 1.4 μM |
| 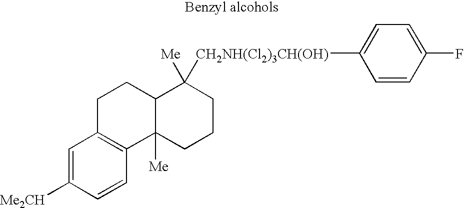 | about 10 μM |
| Benzyl phenyl pyrimidines | |
| 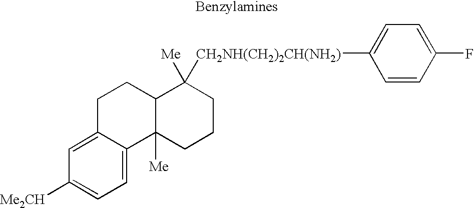 | μM range |
| Cinammic acid compounds | about 70 nM |
| 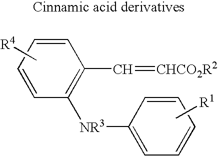 | μM range |
| 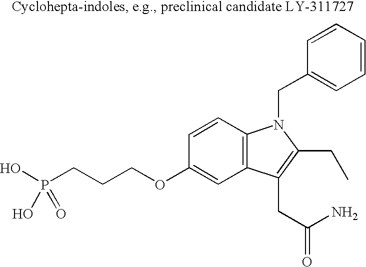 | sub μM range |
| 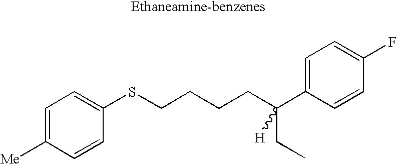 | μM range |
| Imidazolidinones, thiazolidinones and pyrrolidinones | |
| 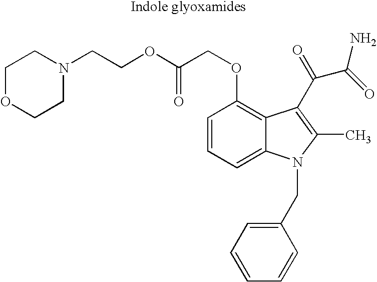 | |
| Indoles | about 0.08 μM to about |
| | 50 μM |
| 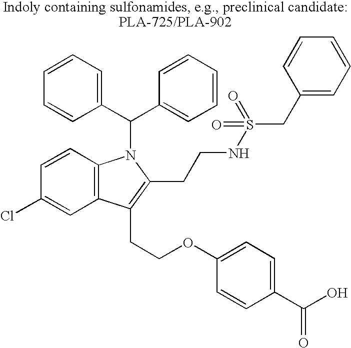 | |
| 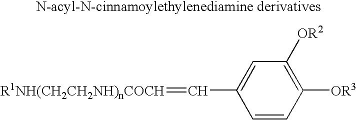 | about 7 μg/mL |
| 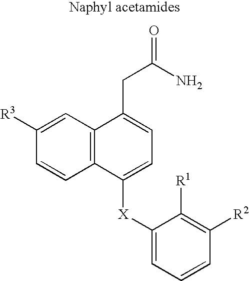 | about 0.87 nμM |
| 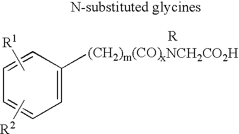 | μM range |
| 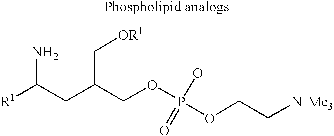 | μM range |
| Piperazines | μM range |
| 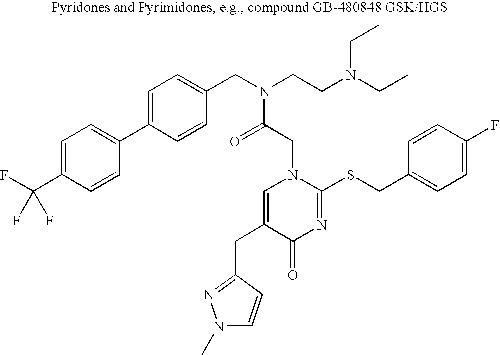 | nM or subnM range |
| 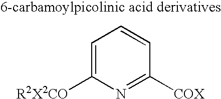 | μM range |
| 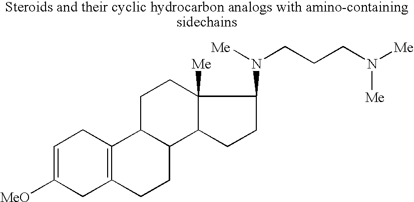 | sub μM range |
| 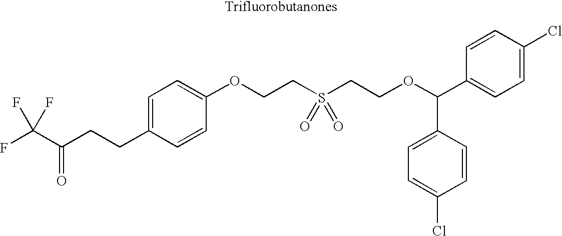 | about 1 μM to about 50 μM |
| 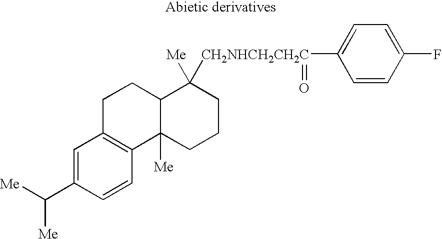 | μM range |
| 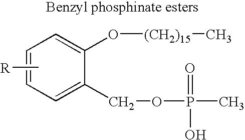 | μM range |
| 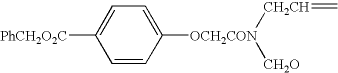 | μM range |

Phospholipase inhibiting moieties useful in some phospholipase inhibitors of the present invention also include natural products, such as Manoalide, a marine product extracted from the sponge Luffariella variabilis, as well as compounds related thereto, illustrated along with the structure of Manoalide below:

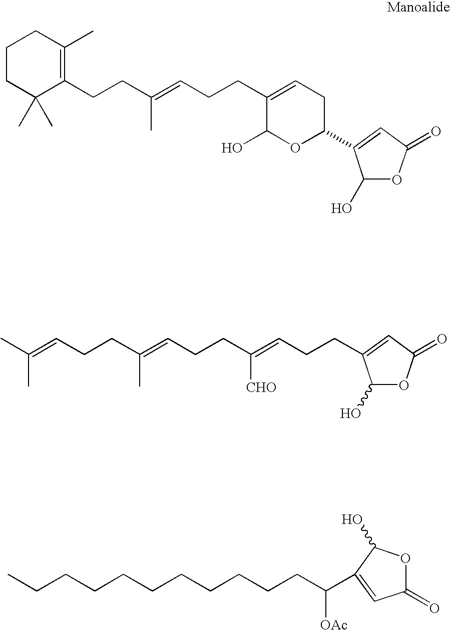

Any of these compounds can be used as a phospholipase inhibiting moiety of the non-absorbed inhibitors in some embodiments of the present invention. As described in more derail above, such moieties may have particular mass, charge and/or other physical parameters to hinder (net) absorption through a gastrointestinal tract, and/or can be linked to a non-absorbed moiety, e.g., a polymer moiety. Furthermore, the invention is not limited to the compositions disclosed herein. Other compositions useful in the present invention would be apparent to one of skill in the art, based on the teachings presented herein, and are also contemplated as within the scope of the invention.

The point of attachment of a phospholipase inhibiting moiety to a non-absorbed moiety, e.g., a polymer moiety, can be selected so as not to interfere with the inhibitory action of the phospholipase inhibiting moiety, e.g., its ability to blunt or reduce the catalytic activity of PLA2. For instance when a phospholipid analog is used as Z, minimal loss of activity can be achieved by attaching the linking moiety to the hydrophobic group of the phospholipid analog (e.g., its long chain alkyl group) rather than, for example, to its polar head group. Without being limited to a particular hypothesis, phospholipid analogs can inhibit PLA2 by competing with phospholipid substrates for the catalytic site, which recognizes the polar head group rather than the hydrophobic group of the phospholipid substrate or phospholipid analog. Thus, attachment to the weakly-recognized hydrophobic group can minimize interference with enzyme inhibitory activity of the phospholipid analog. Those of skill in the art will recognize other suitable attachment points for other art-known phospholipase inhibiting moieties.

For example, suitable points of attachment can be identified by available structural information. A co-crystal structure of a phospholipase inhibiting moiety bound to a phospholipase allows one to select one or more sites where attachment of a linking moiety would not preclude the interaction between the phospholipase inhibiting moiety and its target. For instance, preferred points of attachment of phospholipase inhibiting moieties selected from various classes of art-known phospholipase inhibitors are indicated with arrows below:

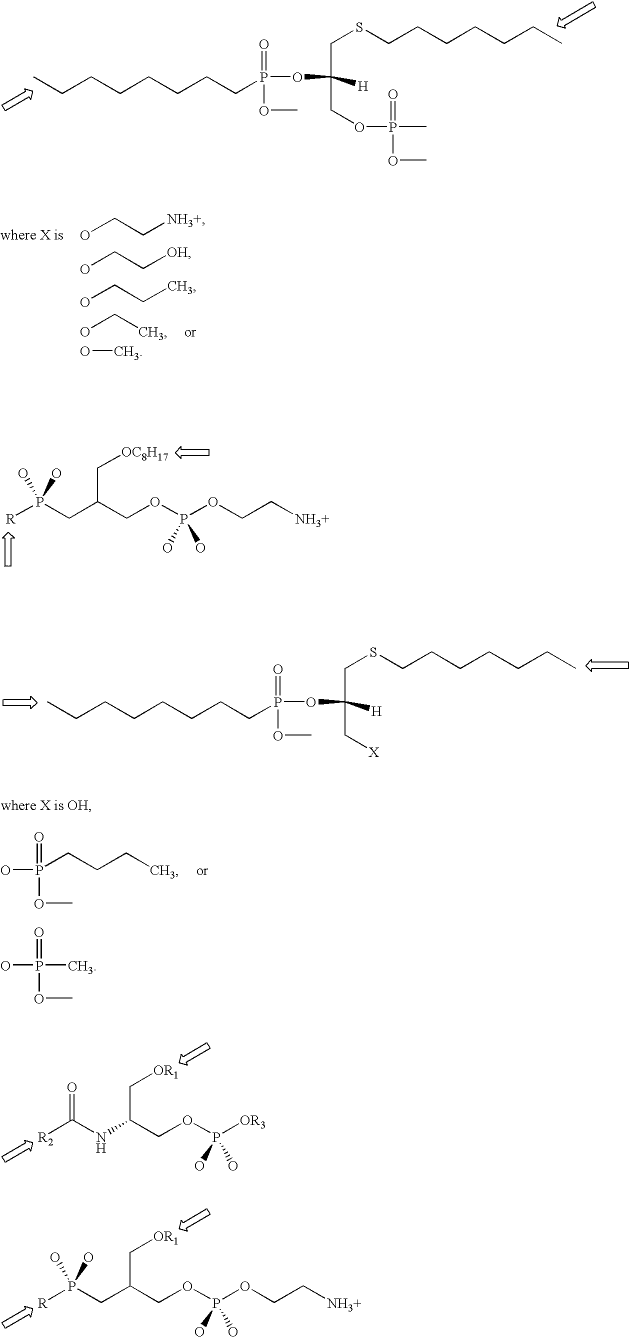

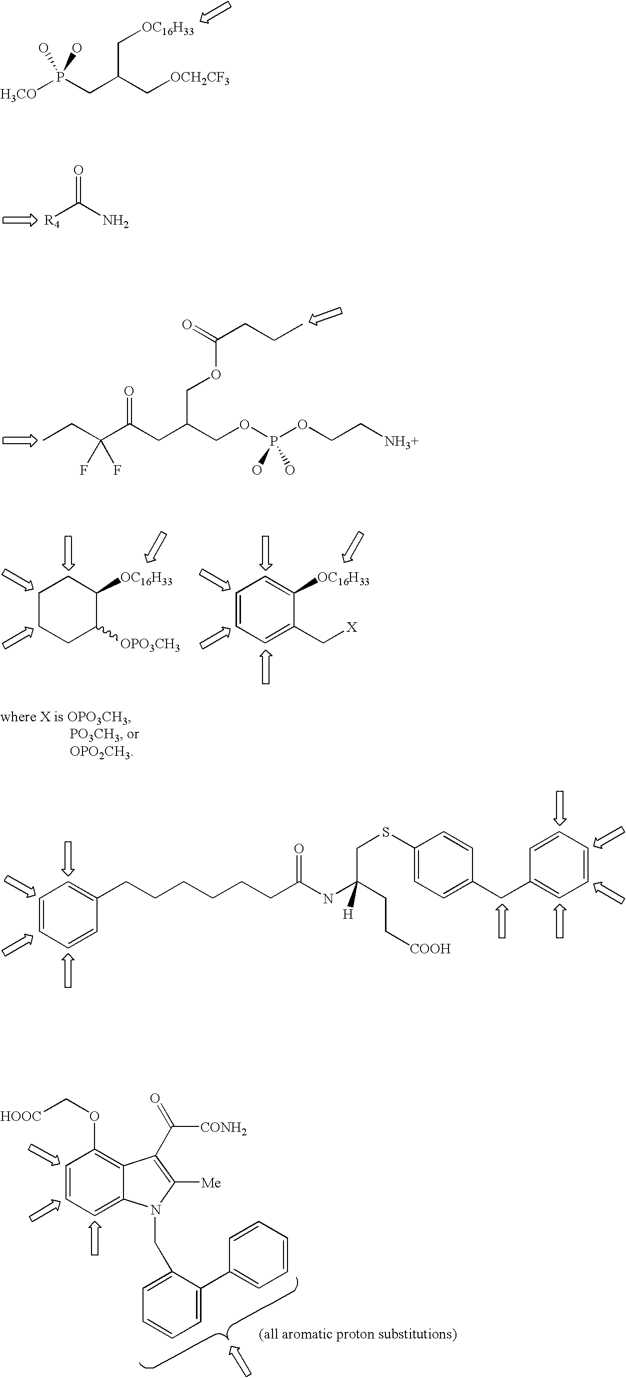

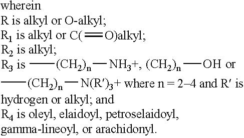

Further, evaluation of binding of a phospholipase inhibitor to a phospholipase by nuclear magnetic resonance permits identification of sites non-essential for such binding interaction. Additionally, one of skill in the art can use available structure-activity relationship (SAR) for phospholipase inhibitors that suggest positions where structural variations are allowed. A library of candidate phospholipase inhibitors can be designed to feature different points of attachment of the phospholipase inhibiting moiety, e.g., chosen based on information described above as well as randomly, so as to present the phospholipase inhibiting moiety in multiple distinct orientations. Candidates can be evaluated for phospholipase inhibiting activity, as discussed in more detail below, to obtain phospholipase inhibitors with suitable attachment points of the phospholipase inhibiting moiety to the polymer moiety or other non-absorbed moiety.

With respect to the polymer moiety, M, a number of polymers can be used including, for example, synthetic and/or naturally occurring aliphatic, alicyclic, and/or aromatic polymers. In preferred embodiments, the polymer moiety is stable under physiological conditions of the GI tract. By “stable” it is meant that the polymer moiety does not degrade or does not degrade significantly or essentially does not degrade under the physiological conditions of the GI tract. For instance, at least about 90%, preferably at least about 95%, and more preferably at least about 98%, and even more preferably at least about 99% of the polymer moiety remains un-degraded or intact after at least about 5 hours, at least about 10 hours, at least about 24 hours, or at least about 48 hours of residence in a gastrointestinal tract. Stability in a gastrointestinal tract can be evaluated using gastrointestinal mimics, e.g., gastric mimics or intestinal mimics of the small intestine, which approximately model the physiological conditions at one or more locations within a GI tract.

The polymer moiety may be soluble or insoluble, existing for example as dispersed micelles or particles, such as colloidal particles or (insoluble) macroscopic beads. In some embodiments, the polymer moiety presents as insoluble porous particles. In preferred embodiments, the polymer moiety is soluble or exists as colloidal dispersions under the physiological conditions of the gastrointestinal tract, for example, at a location within the GI tract where the phospholipase inhibiting moiety acts, e.g., within the gastrointestinal lumen of the small intestine.

Polymer moieties can be hydrophobic, hydrophilic, amphiphilic, uncharged or non-ionic, negatively or positively charged, or a combination thereof, and can be organic or inorganic. Inorganic polymers, also referred to as inorganic carriers in some cases, include silica (e.g., multi-layered silica), diatomeous earth, zheolite, calcium carbonate, talc, and the like.

The polymer architecture of the polymer moiety can be linear, grafted, comb, block, star and/or dendritic, preferably selected to produce desired solubility and/or stability characteristics as described above. The architecture may involve a macromolecular scaffold, and in some embodiments the scaffold may form particles that may be porous or non-porous. The particles may be of any shape, including spherical, elliptical, globular, or irregularly-shaped particles. Preferably the particles are composed of a crosslinked organic polymer derived from, e.g., styrenic, acrylic, methacrylic, allylic, or vinylic monomers, or produced by polycondensation such as polyester, polyamide, melamin and phenol formol condensates, or derived from semi-synthetic cellulose and cellulose-like materials, such as cross-linked dextran or agarose (e.g., Sepharose (Amersham)).

In preferred particle embodiments comprising a phospholipase inhibiting moiety linked, coupled or otherwise attached to a polymer moiety, the particles provide enough available surface area to allow binding of the phospholipase inhibiting moiety to phospholipase. For example, in order to help reduce the dose required to produce a therapeutic and/or a prophylactic benefit, the particles should exhibit specific surface area in the range of about 2 m²/gr to about 500 m²/gr, preferably about 20 m²/gr to about 200 m²/gr, more preferably about 40 m²/gr to about 100 m /gr. Phospholipase inhibiting moieties are preferably linked, coupled or otherwise attached to the polymer moiety on the surface of such particles and preferably at a density of about 0.05 mmol/g to about 4 mmol/g of the polymer moiety, more preferably about 0.1 mmol/g to about 2 mmol/g of the polymer moiety.

In the case where the polymer moiety forms porous particles, beads, or matrices, the pore dimension can be large enough to accommodate phospholipase, e.g., PLA2, within the pores. In some embodiments, for example, porosity may be selected such that the minimum pore size is at least about 2 nm, preferably at least about 5 nm, and more preferably at least about 20 nm. Such materials can be produced by direct or inverse suspension polymerization using process additives such as diluent, porogen, and/or suspension aids, which can control size and porosity.

Polymer moieties useful in constructing non-absorbed inhibitors of the present invention can also be produced by free radical polymerization, condensation, addition polymerization, ring-opening polymerization, and/or can be derived from naturally occurring polymers, such as saccharide polymers. Further, in some embodiments, any of these polymer moieties may be functionalized.

Examples of polysaccharides useful in the present invention include materials from vegetal or animal origin, including cellulose materials, hemicellulose, alkyl cellulose, hydroxyalkyl cellulose, carboxymethylcellulose, sulfoethylcellulose, starch, xylan, amylopectine, chondroitin, hyarulonate, heparin, guar, xanthan, mannan, galactomannan, chitin, and/or chitosan. As noted above, more preferred are polymer moieties that do not degrade or that do not degrade significantly or essentially do not degrade under the physiological conditions of the GI tract, such as carboxymethylcellulose, chitosan, and sulfoethylcellulose.

When free radical polymerization is used, the polymer moiety can be prepared from various classes of monomers including, for example, acrylic, methacrylic, styrenic, vinylique dienic, whose typical examples are given thereafter: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, and combinations thereof. Functionalized versions of these monomers may also be used and any of these monomers may be used with other monomers as comonomers. For example, specific monomers or comonomers that may be used in this invention include methyl methacrylate, ethyl methacrylate, propyl methacrylate (all isomers), butyl methacrylate (all isomers), 2-ethylhexyl methacrylate, isobomyl methacrylate, methacrylic acid, benzyl methacrylate, phenyl methacrylate, methacrylonitrile, α-methylstyrene, methyl acrylate, ethyl acrylate, propyl acrylate (all isomers), butyl acrylate (all isomers), 2-ethylhexyl acrylate, isobomyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, 4-acryloylmorpholine, vinyl benzoic acid (all isomers), diethylaminostyrene (all isomers), a-methylvinyl benzoic acid (all isomers), diethylamino α-methylstyrene (all isomers), p-vinylbenzene sulfonic acid, p-vinylbenzene sulfonic sodium salt, alkoxy and alkyl silane functional monomers, maleic anhydride, N-phenylmaleimide, N-butylmaleimide, butadiene, isoprene, chloroprene, ethylene, vinyl acetate, vinylformamide, allylamine, vinylpyridines (all isomers), fluorinated acrylate, methacrylates, and combinations thereof. Main chain heteroatom polymer moieties can also be used, including polyethyleneimine and polyethers such as polyethylene oxide and polypropylene oxide, as well as copolymers thereof.

In some embodiments a phospholipase inhibitor is constructed to hinder its (net) absorption through a gastrointestinal mucosa and/or comprises a phospholipase inhibiting moiety linked, coupled or otherwise attached to a non-absorbed moiety as described above. In some embodiments, the phospholipase inhibitor is localized in a gastrointestinal lumen due to efflux. In some embodiments, the inhibitor is effluxed from a gastrointestinal mucosal cell, for example, an intestinal and/or a colonic enterocyte, upon entry into the cell, creating the net effect of non-absorption. Any art-known phospholipase inhibitor and/or any phospholipase inhibiting moiety described and/or contemplated herein can be used in these embodiments. For example, any art known PLA2 inhibitors provided in Table 1 can be used. These and other art-known phospholipase inhibitors and/or any phospholipase inhibiting moiety disclosed and/or contemplated herein can be constructed to be effluxed back into a gastrointestinal lumen upon movement therefrom.

In some efflux embodiments, the phospholipase inhibitor remains localized in the gastrointestinal lumen even though it may be absorbed by a gastrointestinal mucosal cell by active and/or passive transport, or otherwise permeate through the gastrointestinal wall by active and/or passive transport. The phospholipase inhibitor in some embodiments may have one or more hydrophobic and/or lipophilic moieties, tending to allow diffusion across the plasma membrane of a gastrointestinal mucosal cell. However, subsequent passage across the basolateral membrane and into the portal blood circulation can be regulated by a number of physical and molecular considerations, discussed in detail below. For example, a phospholipase inhibitor that enters an intestinal and/or a colonic enterocyte, e.g., an apical enterocyte, can be subsequently effluxed back into the gastrointestinal lumen.

In some embodiments, efflux is achieved by protein and/or glycoprotein transporters located in a gastrointestinal mucosal cell, for example, in an apical enterocyte of the gastrointestinal tract. Protein and/or glycoprotein transporters include, but are not limited to, for example, ATP-binding cassette transport proteins, such as P-glycoproteins including MDR1 (product of ABCB1 locus) and MRP2, located in the epithelial cells of the gut, for example, in the apical enterocytes of the gastrointestinal tract. Such transports may also be referred to pumps.

In some embodiments, for example, a phospholipase inhibitor can be constructed so as to be recognized by a protein and/or glycoprotein transporter that effluxes the inhibitor from the cytoplasm of an enterocyte back into the gastrointestinal lumen. In some embodiments, the phospholipase inhibitor is constructed so as to allow intracellular modification, e.g., via metabolic processes, within the enterocyte to facilitate recognition by a protein and/or glycoprotein transporter, such that the modified inhibitor serves as a target for transport. Motifs that are recognized by protein and/or glycoprotein transporters of the gut epithelium can be determined by one of ordinary skill in the art. For example, recognition motifs for ATP-binding cassette transport proteins, such as P-glycoproteins including MDR1 (product of ABCB1 locus) and MRP2 can be determined. A phospholipase inhibitor of the present invention may comprise a phospholipase inhibiting moiety linked, coupled, or otherwise attached to a recognition motif moiety. “Recognition motif moiety” as used herein refers to a moiety comprising a motif that is recognized by a transporter, or than can be modified to become recognized by a transporter, where the transporter can effect efflux of a composition comprising the recognition motif moiety into the gastrointestinal lumen, including, for example motifs recognized by protein and/or glycoprotein transporters of the gut epithelium such as ATP-binding cassette transport proteins, P-glycoproteins, MDR1, MRP2, and the like. In some embodiments, the recognition motif moiety serves as a target for a transporter of a gut epithelial cell, causing the transporter to drive the phospholipase inhibitor from the inside of the cell back into the gastrointestinal lumen. Lumen localization achieved by efflux can thus hinder or prevent absorption of the phospholipase inhibitor into the blood circulation.

In preferred embodiments, efflux achieves lumen localization of a significant amount, preferably a statistically significant amount, and more preferably essentially all, of the phospholipase inhibitor introduced into the gastrointestinal lumen. That is, essentially all of the phospholipase inhibitor remains in the gastrointestinal lumen by efflux of some, most, and/or essentially all of any inhibitor that moves out of the gastrointestinal lumen. For example, the effect can be such that at least about 90% of phospholipase inhibitor remains in the gastrointestinal lumen, at least about 95%, at least about 98%, preferably at least about 99%, and more preferably at least about 99.5% remains in the gastrointestinal lumen.

In some embodiments, the phospholipase inhibitor comprises one or more additional efflux enhancing moieties. “Efflux enhancing moiety” as used herein refers to a moiety comprising an efflux enhancer that acts to enhance, aid, increase, activate, promote, or otherwise facilitate efflux of the moiety into the gastrointestinal lumen. For example, the phospholipase inhibitor in some embodiments may comprise a moiety that activates expression of a transporter, for example, a transcription factor and/or an enhancer of a gene encoding a transporter. For example, the nuclear receptor, pregnane X, also referred to as the pregnane X receptor (PXR), induces high levels of MDR1 and/or related transporters. (CITE). In some preferred embodiments, the phospholipase inhibitor is coupled, linked and/or otherwise attached to an efflux enhancing moiety that activates PXR, e.g., by contacting and binding to the nuclear receptor. The higher levels of MDR1 and/or related transporters produced can enhance efflux of phospholipase inhibitor that also comprises, for example, a recognition motif for MDR1. Based on the teachings herein, those of ordinary skill in the art will recognize other efflux enhancing moieties that may be used in these aspects of the invention, and which are also contemplated within its scope.

Some embodiments of the present invention involve a combination of non-absorbed and effluxed inhibitors. In such embodiments, lumen localization is achieved by a combination of non-absorption of the phospholipase inhibitor and efflux of some, most, and/or essentially all of any phospholipase inhibitor that moves out of the gastrointestinal lumen.

Lumen-localization can improve the potency of the phospholipase inhibitor, so that the amount of inhibitor administered can be less than the amount administered in the absence of non-absorption and/or efflux. In some embodiments, non-absorption and/or efflux improves the efficacy of the phospholipase inhibitor. In particular, the inhibitor reduces the activity of phospholipase to a greater extent when localized in the lumen by non-absorption and/or efflux. In such embodiments, the amount of phospholipase inhibitor used can be the same as the recommended dosage levels or higher than this dose or lower than the recommended dose. In some embodiments, non-absorption and/or efflux decreases the dose of phospholipase inhibitor used and thus can increase patient compliance and decrease side-effects.

Phospholipase Inhibition by Lumen-Localized Phospholipase Inhibitors

The present invention provides compositions comprising a phospholipase inhibitor that is not absorbed through a gastrointestinal mucosa and/or that is localized in a gastrointestinal lumen as a result of efflux from a gastrointestinal mucosal cell.

In some embodiments, a phospholipase inhibitor of the present invention acts by hindering access of the enzyme to its phospholipid substrate; in some embodiments it acts by reducing the enzyme's catalytic activity with respect to its substrate; in some embodiments the phospholipase inhibitor acts by a combination of these two approaches.

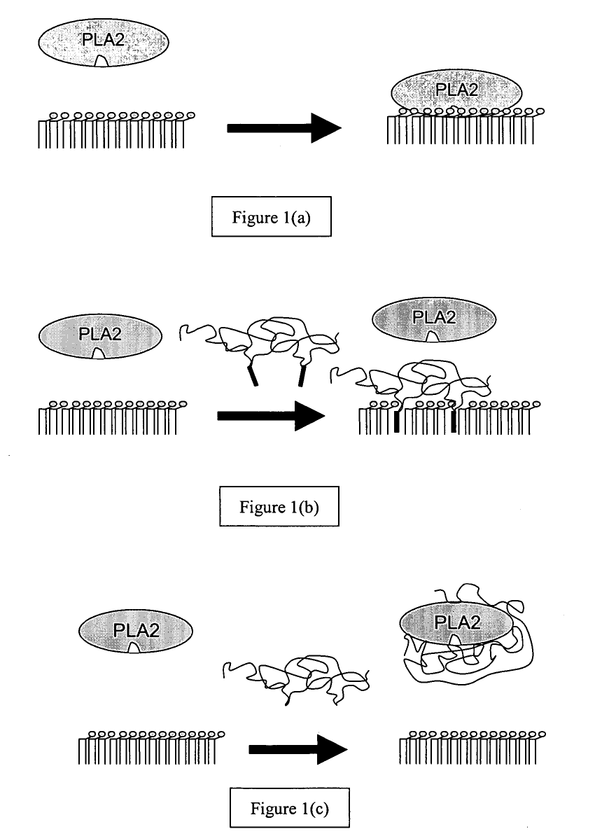

As discussed above, some gastrointestinal phospholipases, e.g., most PLA2 enzymes, act on their substrates while “docked” to a lipid-water interface of a lipid aggregate, meaning that catalytic activity depends on the enzyme's physical access to the outer surface of lipid aggregates in the gastrointestinal lumen. This interaction is depicted diagrammatically in FIG. 1(a). This figure provides an illustration only, and is in no way intended to be limiting with respect to the present invention. For example, those of ordinary skill in the art will readily appreciate variations and modifications of the scheme illustrated. The figure diagrammatically depicts PLA2 interacting with a lipid-water interface of a lipid aggregate. The catalytic site of the i-face of the enzyme is depicted by a “notch” on the face that interacts with the lipid aggregate.

In some embodiments of the present invention, PLA2 inhibition is achieved by keeping the enzyme off the outer surface of lipid aggregates, thereby hindering access to phospholipid substrates. FIGS. 1(b) and (c) illustrate two embodiments of non-absorbed phospholipase inhibitors that act by hindering access to a phospholipid substrate at a lipid-water interface.

A non-absorbed inhibitor that acts by hindering access need not directly interfere with the catalytic site of the enzyme, for example, it need not recognize and/or bind to the enzyme's catalytic site or to any other specific site on the enzyme, such as an allosteric site. Rather, in some embodiments, a non-absorbed phospholipase inhibitor of the present invention may prevent or hinder physical adsorption of the enzyme at a lipid-water interface of one or more types of lipid aggregates found in the gastrointestinal lumen. Examples of a “lipid-water interface” include the outer surface of a lipid aggregate found in the gastrointestinal lumen, including, for example, a fat globule, an emulsion droplet, a vesicle, a mixed micelle, and/or a disk, any one of which may contain triglycerides, fatty acids, bile acids, phospholipids, phosphatidylcholine, lysophospholipids, lysophosphatidylcholine, cholesterol, cholesterol esters, other amphiphiles and/or other diet metabolites.

In preferred embodiments, the inhibitor comprises a polymer moiety capable of interacting with either a phospholipase and/or the lipid-water interface of a lipid aggregate. FIG. 1(a) illustrates the situation where the inhibitor interacts with a lipid-water interface such that it becomes physically complexed, coupled, bound, attached, or otherwise adsorbed to the lipid-water interface. This figure provides an illustration only, and is in no way intended to be limiting with respect to the present invention. For example, those of ordinary skill in the art will readily appreciate variations and modifications of the scheme illustrated, in light of the disclosure provided herein, and such variations and modifications are contemplated as within the scope of the present invention. The inhibitor can interact with the interface through any bonding interaction, including, for example, covalent, ionic, metallic, hydrogen, hydrophobic, and/or van der Waals bonds, preferably hydrophobic an/or ionic bonds. FIG. 1(b) illustrates a situation where inhibitor interaction with a lipid-water interface is facilitated by hydrophobic bonds. In this depicted embodiment, the inhibitor bears two hydrophopic moieties (depicted by solid rectangles), e.g., phospholipid analogs, that become embedded in the lipid layer via hydrophobic interactions between the moieties of the inhibitor and the hydropholonic chains of the layer.

FIG. 1(c) illustrates the situation where the inhibitor interacts with a phospholipase enzyme, e.g. PLA2. This figure provides an illustration only, and is in no way intended to be limiting with respect to the present invention. For example, those of ordinary skill in the art will readily appreciate variations and modifications of the scheme illustrated, in light of the disclosure provided herein, and such variations and modifications are contemplated as within the scope of the present invention. In some embodiments, the phospholipase inhibitor comprises a moiety that becomes physically complexed, coupled, bound, attached, or otherwise adsorbed to the enzyme so as to hinder its interaction with a lipid aggregate. The inhibitor can be described as scavenging the enzyme in solution to create a complex with it. In some embodiments, the enzyme interacting with the inhibitor is sterically hindered from access to its phospholipid substrate at a lipid-water interface, for example, because its approach to the interface is physically hindered.

In some embodiments, the inhibitor comprises a polymer moiety that can be soluble or insoluble under the physiological conditions of the gastrointestinal lumen, and may exist, for example, as dispersed micelles or particles, such as colloidal particles or (insoluble) macroscopic beads, as described in detail above. The polymer moiety of the inhibitor can be shaped in various formats, preferably designed to favor the formation of a complex with a phospholipase, e.g., a complex with PLA2. For instance, the polymer moiety may comprise a macromolecular scaffold designed to interact with the i-face of PLA2. As discussed above, the structural features of the i-face are such that the aperture of the slot forming the catalytic site is normal to the i-face plane. The aperture is surrounded by a first crown of hydrophobic residues (mainly leucine and isoleucine residues), which itself is contained in a ring of cationic residues, (including lysine and arginine residues). The polymer moiety may be designed as a macromolecular scaffold comprising a plurality of anionic moieties (e.g., arranged so as to bind to the cationic ring) and/or a plurality of hydrophobic residues (e.g., arranged so as to bind to the hydrophobic crown). In such embodiments, the inhibitor becomes positioned over the catalytic site bearing face of a phospholipase and hinders access to the catalytic site as a “lid” or “cap” blocks access to an aperture.

In some embodiments, the inhibitor also comprises a phospholipase inhibiting moiety, for example any art-known phospholipase inhibitor and/or any of the phospholipase inhibiting moiety described and/or contemplated herein. The phospholipase inhibiting moiety may be coupled, linked or otherwise attached to a non-absorbed moiety, including, for example, a polymer moiety that interacts with a lipid-water interface and/or a polymer moiety that interacts with phospholipase. In the latter case, the phospholipase inhibiting moiety may further aid the interaction of the polymer moiety with the phospholipase, e.g., with the i-face of PLA2.

In some embodiments, for example, a PLA2 inhibiting is moiety linked, coupled or otherwise attached is coupled to a macromolecular scaffold of a polymer moiety where the PLA2 inhibiting moiety interacts with the catalytic site of PLA2 while the macromolecular scaffold interacts with the i-face surrounding the catalytic site. Where the phospholipase inhibiting moiety comprises a phospholipid analog or a transition state analog, the phospholipase inhibiting moiety is preferably coupled via its hydrophobic group, leaving the polar head group of the inhibiting moiety available for binding to the catalytic site, e.g., through the His-calcium-Asp triad discussed above.

Some embodiments comprising a phospholipase inhibiting moiety coupled to a polymer moiety that interacts with a phospholipase comprise a plurality of anionic moieties (e.g., arranged so as to bind to a cationic ring) linked to a spacer moiety (e.g., arranged so as to overlay a hydrophobic crown), which converge on a central or focal point bearing the phospholipase inhibiting moiety. Some such embodiments can be represented by the following formula: 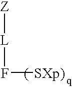
where Z is a phospholipase inhibiting moiety, preferably a PLA2 inhibiting moiety; L is a linking moiety, e.g., a chemical linker; F is focal point where covalent linkages from a plurality of segments SXp converge; S is a spacer moiety; X is an anionic moiety, preferably an acidic group, for example, but not limited to, a carboxylate group, a sulfonate group, a sulfate group, a sulfamate group, a phosphoramidate group, a phosphate group, a phosphonate group, a phosphinate group, a gluconate group, and the like; and p and q are each integers, preferably where p equals 1, 2, 3, or 4, and preferably where q equals 2, 3, 4, 5, 6, 7, or 8.

The F-(SXp)q segment can adopt various configurations, preferably configurations that facilitate interaction with the catalytic site bearing face of a phospholipase. In some embodiments, for example, a plurality of spacer moieties radiate from the focal point F, which lies at a center of a macromolecular scaffold of the polymer moiety;

In some preferred embodiments, the spacer moiety S provides a plurality of hydrophobic residues, e.g., arranged so as to bind to the hydrophobic crown of the i-face of PLA2; in some preferred embodiments, the anionic moieties X are arranged so as to bind to the cationic ring of the i-face of PLA2. Some embodiments comprise a dendritic macromolecular scaffold with spacer moieties branching and diverging from the focal point F. Examples of some embodiments can be represented by the structures provided below:

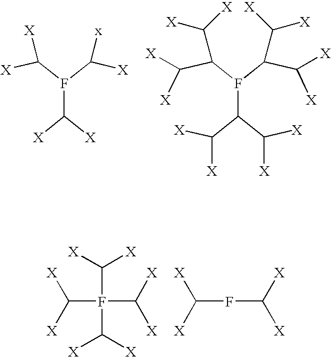

Other examples of dendritic structures useful in the practice of the present invention are known in the art, e.g., see Grayson S. M. et al. Chemical Reviews, 2001, 101: 3819-3867; and Bosman A. W. et al, Chemical Reviews, 1999, 99; 1665-1688, incorporated herein by reference. Additionally, other examples suitable for use in the present invention will be appreciated by those of ordinary skill in the art in light of the disclosures provided herein, and are contemplated as within the scope of this invention.

In some embodiments, the macromolecular scaffold of the polymer moiety can form particles. In such embodiments, a phospholipase inhibiting moiety is preferably coupled to the outer surfaces of such particles. Where the phospholipase inhibiting moiety is a phospholipid analog or transition state analog, the phospholipase inhibiting moiety is preferably linked through its hydrophobic group, as discussed above. The particles so formed may be porous or non-porous, and may be of any shape, such as spherical, elliptical, globular, or irregularly-shaped particles, as discussed in more detail above. The particles can be composed of one or more organic or inorganic polymers moieties, including any of the polymers disclosed herein. In preferred particle embodiments, the particle surface is hydrophobic in nature, carrying acidic groups, X as defined above.

In other embodiments where non-absorbed phospholipase inhibitors comprise a moiety interacting with a specific site on a phospholipase, e.g., the catalytic site of PLA2, the inhibitor need not prevent access of the enzyme to its substrate, but may act by reducing the enzyme's ability to act on its substrate even if the enzyme approaches and/or becomes “docked” to a lipid-water interface containing the substrate. Such inhibitor embodiments preferably comprise a polymer moiety and one or more phospholipase inhibiting moieties, e.g., an art-known phospholipase inhibitor and/or any phospholipase inhibitor described and/or contemplated herein. Without being bound to a particular hypothesis, for example, such inhibitors can act to reduce phospholipase activity by reversible and/or irreversible inhibition.

Reversible inhibition by a phospholipase inhibitor of the present invention may be competitive (e.g. where the inhibitor binds to the catalytic site of a phospholipase), noncompetitive (e.g., where the inhibitor binds to an allosteric site of a phospholipase to effect an allosteric change), and/or uncompetitive (where the inhibitor binds to a complex between a phospholipase and its substrate). Inhibition may also be irreversible, where the phospholipase inhibitor remains bound, or significantly remains bound, or essentially remains bound to a site on a phospholipase without dissociating, without significantly dissociating, or essentially without dissociating from the enzyme.

As discussed above, PLA2 enzymes share a conserved active site architecture and a catalytic mechanism involving concerted binding of His and Asp residues to water molecules and a calcium cation. Phospholipid substrate can access the catalytic site by its polar head group through a slot enveloped by hydrophobic and cationic residues. Within the catalytic site, the multi-coordinated calcium ion activates the acyl carbonyl group of the sn-2 position of the phospholipid substrate to bring about hydrolysis. In certain embodiments, PLA2 inhibiting moieties comprise structures that resemble a phospholipid substrate and/or its transition state.

Without being limited to a particular hypothesis, such moieties can inhibit PLA2 by competing reversibly with phospholipid substrates for the catalytic site. That is, a structural analog of a phospholipid substrate, preferably, a structural analog of its polar head group and/or a structural analog of a phospholipid substrate transition state can reversibly bind the catalytic site, inhibiting access of the phospholipid substrate. Further, as described in detail above, analog phospholipase inhibiting moieties can be attached to a non-absorbed moiety, e.g., a polymer moiety, at an attachment point that does not interfere with the ability of the analog to bind to the catalytic site, minimizing the inhibitory activity of the analog.

Further, in some of these embodiments, the phospholipase inhibitor reduces re-absorption of secreted phospholipase A2 through the gastrointestinal mucosa.

Screening Assays for Identifying Phospholipase Inhibitors

The differential activities of gastrointestinal phospholipases, in particular phospholipase A2, enables the screening for inhibitory compounds that inhibit a particular phospholipase and that can be used with the practice of this invention to selectively treat insulin-related conditions (e.g., diabetes), weight-related conditions (e.g., obesity), cholesterol-related conditions, or a combination thereof.

Inhibitors of Phospholipase A2

Certain aspects of the present invention provide a method of making a phospholipase inhibitor that is localized in a gastrointestinal lumen involving selecting a moiety that inhibits PLA2 by contacting a candidate moiety with a PLA2 enzyme or fragment thereof, preferably a fragment containing the catalytic and/or allosteric site of the enzyme, more preferably including the His and Asp residues of the catalytic site; determining whether the candidate moiety interacts with the PLA2 or fragment thereof; and using the selected candidate moiety as a phospholipase A2 inhibiting moiety of a phospholipase inhibitor that is localized in a gastrointestinal lumen.

Certain other aspects of the present invention provide a method of making a phospholipase inhibitor that is localized in a gastrointestinal lumen involving selecting a moiety that inhibits PLA2 by contacting a candidate moiety with a lipid-water interface of a lipid aggregate or fragment thereof; determining whether the candidate moiety interacts with the interface; and using the selected candidate moiety as a phospholipase A2 inhibiting moiety of a phospholipase inhibitor that is localized in a gastrointestinal lumen.

Inhibitors of Phospholipase B

Certain aspects of the present invention provide a method of making a phospholipase inhibitor that is localized in a gastrointestinal lumen involving selecting a moiety that inhibits PLB by contacting a candidate moiety with a PLB enzyme or fragment thereof; determining whether the candidate moiety interacts with the PLB or fragment thereof; and using the selected candidate moiety as a phospholipase B inhibiting moiety of a phospholipase inhibitor that is localized in a gastrointestinal lumen.

Inhibitors of Phospholipase A2 but not of Phospholipase B

Certain aspects of the present invention provide a method of making a phospholipase inhibitor that is localized in a gastrointestinal lumen involving selecting a moiety that preferentially inhibits PLA2 by contacting a candidate moiety with a PLA2 enzyme or fragment thereof, preferably a fragment containing the catalytic and/or allosteric site of the enzyme, more preferably including the His and Asp residues of the catalytic site and determining whether the candidate moiety interacts with the PLA2 or fragment thereof; contacting the candidate with a PLB enzyme or fragment thereof and determining whether the candidate interacts with the PLB or fragment thereof; selecting any candidate that interacts with PLA2 but does not interact with PLB, does not significantly interact with PLB, or essentially does not interact with PLB; and using the selected candidate moiety as a phospholipase A2 inhibiting moiety of a phospholipase inhibitor that is localized in a gastrointestinal lumen.

Certain other aspects of the present invention provide a method of making a phospholipase inhibitor that is localized in a gastrointestinal lumen involving selecting a moiety that preferentially inhibits PLA2 by contacting a candidate with a lipid-water interface of a lipid aggregate or fragment thereof and determining whether the candidate moiety interacts with the interface; contacting the candidate moiety with a PLB enzyme or fragment thereof and determining whether the candidate moiety interacts with the PLB or fragment thereof; selecting any candidate moiety that interacts with the lipid-water interface does not interact with PLB, but does not significantly interact with PLB, or essentially does not interact with PLB, and using the selected candidate moiety as a phospholipase A2 inhibiting moiety of a phospholipase inhibitor that is localized in a gastrointestinal lumen.

A lumen-localized phospholipase inhibitor, for example, comprising a phospholipase inhibiting moiety disclosed herein and/or identified by the procedures taught herein, can be used in animal models to demonstrate, for example, suppression of insulin-related conditions (e.g. diabetes) and/or hypercholesterolemia and/or weight-related conditions. A lumen-localized phospholipase inhibitor showing inhibitory activity in a PLA2 inhibition assay, in about the sub μM range is preferred. More preferably, such inhibitors show non-absorbedness, for example low permeability, in any assays disclosed herein or known in the art. Examples of suitable animal models are described in more detail below.

Non-absorbed and/or effluxed phospholipase inhibitors of the present invention can form the basis of pharmaceutical compositions and kits that find use in methods of treating a subject by administering the composition. Preferably, such compositions modulate the activity of a gastrointestinal phospholipase, for example, reducing the activity of phospholipase A2 and/or one or more other phospholipases. In some embodiments, the phospholipase inhibitor inhibits phospholipase A2. In some embodiments, the phospholipase inhibitor inhibits phospholipase A2 and phospholipase B. In some embodiments, the phospholipase inhibitor inhibits phospholipase A2 but does not inhibit or does not significantly inhibit or essentially does not inhibit phospholipase B. In some embodiments, the phospholipase inhibitor inhibits phospholipase A2 but does not inhibit or does not significantly inhibit or essentially does not inhibit other gastrointestinal phospholipases.

Methods of Treating Phospholipase-Related Conditions

The present invention provides methods of treating phospholipase-related conditions where the inhibitor is localized in a gastrointestinal lumen. The term “phospholipase-related condition” as used herein refers to a condition in which modulating the activity and/or re-absorption of a phospholipase, and/or modulating the production and/or effects of one or more products of the phospholipase, is desirable. In preferred embodiments, an inhibitor of the present invention reduces the activity and/or re-absorption of a phospholipase, and/or reduces the production and/or effects of one or more products of the phospholipase. The term “phospholipase A2-related condition” as used herein refers to a condition in which modulating the activity and/or re-absorption of phospholipase A2 is desirable and/or modulating the production and/or effects of one or more products of phospholipase A2 activity is desirable. In preferred embodiments, an inhibitor of the present invention reduces the activity and/or re-absorption of phospholipase A2, and/or reduces the production and/or effects of one or more products of the phospholipase A2. Examples of phospholipase A2-related conditions include, but are not limited to, insulin-related conditions (e.g., diabetes), weight-related conditions (e.g., obesity) and/or cholesterol-related conditions, and any combination thereof.

The present invention provides methods, pharmaceutical compositions, and kits for the treatment of animal subjects. The term “animal subject” as used herein includes humans as well as other mammals.

The term “treating” as used herein includes achieving a therapeutic benefit and/or a prophylactic benefit. By therapeutic benefit is meant eradication or amelioration of the underlying disorder being treated. For example, in a diabetic patient, therapeutic benefit includes eradication or amelioration of the underlying diabetes. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, with respect to diabetes reducing PLA2 activity can provide therapeutic benefit not only when insulin resistance is corrected, but also when an improvement is observed in the patient with respect to other disorders that accompany diabetes like fatigue, blurred vision, or tingling sensations in the hands or feet. For prophylactic benefit, a phospholipase inhibitor of the present invention may be administered to a patient at risk of developing a phospholipase-related condition, e.g., diabetes, obesity, or hypercholesterolemia, or to a patient reporting one or more of the physiological symptoms of such conditions, even though a diagnosis may not have been made.

The present invention provides compositions comprising a phospholipase inhibitor that is not absorbed through a gastrointestinal mucosa and/or that is localized in a gastrointestinal lumen as a result of efflux from a gastrointestinal mucosal cell. In preferred embodiments, the phospholipase inhibitors of the present invention produce a benefit, including either a prophylactic benefit, a therapeutic benefit, or both, in treating one or more conditions by inhibiting phospholipase activity.

The term “inhibits” and its grammatical variations are not intended to require a complete inhibition of enzymatic activity. For example, it can refer to a reduction in enzymatic activity by at least about 50%, at least about 75%, preferably by at least about 90%, more preferably at least about 98%, and even more preferably at least about 99% of the activity of the enzyme in the absence of the inhibitor. Most preferably, it refers to a reduction in enzyme activity by an effective amount that is by an amount sufficient to produce a therapeutic and/or a prophylactic benefit in at least one condition being treated. in a subject receiving phospholipase inhibiting treatment, e.g., as disclosed herein. Conversely, the phrase “does not inhibit” and its grammatical variations does not require a complete lack of effect on the enzymatic activity. For example, it refers to situations where there is less than about 20%, less than about 10%, less than about 5%, preferably less than about 2%, and more preferably less than about 1% of reduction in enzyme activity in the presence of the inhibitor. Most preferably, it refers to a minimal reduction in enzyme activity such that a noticeable effect is not observed. Further, the phrase “does not significantly inhibit” and its grammatical variations refers to situations where there is less than about 40%, less than about 30%, less than about 25%, preferably less than about 20%, and more preferably less than about 15% of reduction in enzyme activity in the presence of the inhibitor. Further, the phrase “essentially does not inhibit” and its grammatical variations refers to situations where there is less than about 30%, less than about 25%, less than about 20%, preferably less than about 15 %, and more preferably less than about 10% of reduction in enzyme activity in the presence of the inhibitor.

The methods for effectively inhibiting phospholipase described herein can apply to any phospholipase-related condition, that is, to any condition in which modulating the activity and/or re-absorption of a phospholipase, and/or modulating the production and/or effects of one or more products of the phospholipase, is desirable. Preferably, such conditions include phospholipase A2-related conditions and/or phospholipase A2-related conditions induced by diet, that is, conditions which are brought on, accelerated, exacerbated, or otherwise influenced by diet. Phospholipase A2-related conditions include, but are not limited to, diabetes, weight gain, and cholesterol-related conditions, as well as hyperlipidemia, hypercholesterolemia, cardiovascular disease (such as heart disease and stroke), hypertension, cancer, sleep apnea, osteoarthritis, gallbladder disease, fatty liver disease, diabetes type 2 and other insulin-related conditions. In some embodiments, one or more of these conditions may be produced as a result of consumption of a high fat or Western diet; in some embodiments, one or more of these conditions may be produced as a result of genetic causes, metabolic disorders, environmental factors, behavioral factors, or any combination of these.

Treatment of Insulin-Related Conditions

The term “insulin-related disorders” as used herein refers to a condition such as diabetes where the body does not produce and/or does not properly use insulin. Typically, a patient is diagnosed with pre-diabetes or diabetes by using a Fasting Plasma Glucose Test (FPG) and/or an Oral Glucose Tolerance Test (OGTT). In the case of the FPG test, a fasting blood glucose level between about 100 and about 125 mg/dl can indicate pre-diabetes; while a person with a fasting blood glucose level of about 126 mg/dl or higher can indicate diabetes. In the case of the OGTT test, a patient's blood glucose level can be measured after a fast and two hours after drinking a glucose-rich beverage. A two-hour blood glucose level between about 140 and about 199 mg/dl can indicate pre-diabetes; while a two-hour blood glucose level at about 200 mg/dl or higher can indicate diabetes.

In certain embodiments, a lumen localized phospholipase inhibitor of the present invention produces a benefit in treating an insulin-related condition, for example, diabetes, preferably diabetes type 2. For example, such benefits may include, but are not limited to, increasing insulin sensitivity and improving glucose tolerance. Other benefits may include decreasing fasting blood insulin levels, increasing tissue glucose levels and/or increasing insulin-stimulated glucose metabolism.

Without being limited to any particular hypothesis, these benefits may result from a number of effects brought about by reduced PLA2 activity, including, for example, reduced membrane transport of phospholipids across the gastrointestinal mucosa and/or reduced production of 1-acyl lysophospholipids, such as 1-acyl lysophosphatydylcholine and/or reduced transport of lysophospholipids, 1-acyl lysophosphatydylcholine, that may act as a signaling molecule in subsequent pathways involved in diabetes or other insulin-related conditions.

In some embodiments, a lumen-localized phospholipase inhibitor is used that inhibits phospholipase A2 but does not inhibit or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase.

Treatment of Weight-Related Conditions

The term “weight-related conditions” as used herein refers to unwanted weight gain, including overweight, obese and/or hyperlipidemic conditions, and in particular weight gain caused by a high fat or Western diet. A “high fat” diet includes, for example, diets high in meat, dairy products, and alcohol, as well as possibly including processed food stuffs, red meats, soda, sweets, refined grains, deserts, and high-fat dairy products, for example, where at least about 25% of calories come from fat and at least about 8% come from saturated fat; or at least about 30% of calories come from fat and at least about 10% come from saturated fat; or where at least about 34% of calories came from fat and at least about 12% come from saturated fat; or where at least about 42% of calories come from fat and at least about 15% come from saturated fat; or where at least about 50% of calories come from fat and at least about 20% come from saturated fat. One such high fat diet is a “Western diet” which refers to the diet of industrialized countries, including, for example, a typical American diet, Western European diet, Australian diet, and/or Japanese diet. One particular example of a Western diet comprises at least about 17% fat and at least about 0.1% cholesterol (wt/wt); at least about 21% fat and at least about 0.15% cholesterol (wt/wt); or at least about 25% and at least about 0.2% cholesterol (wt/wt).

Typically, body mass index (BMI) is used as the criteria in determining whether an individual is overweight and/or obese. An adult is considered overweight if, for example, he or she has a body mass index of at least about 25, and is considered obese with a BMI of at least about 30. For children, charts of Body-Mass-Index for Age are used, where a BMI greater than about the 85th percentile is considered “at risk of overweight” and a BMI greater than about the 95th percentile is considered “obese.”

In certain embodiments, a lumen localized phospholipase A2 inhibitor of the present invention can be used to treat weight-related conditions, including unwanted weight gain and/or obesity. In certain embodiments, a lumen localized phospholipase A2 inhibitor decreases fat absorption after a meal typical of a Western diet. In certain embodiments, a lumen localized phospholipase A2 inhibitor increases lipid excretion from a subject on a Western diet. In certain preferred embodiments, the phospholipase inhibitor reduces weight gain in a subject on a (typical) Western diet. In certain embodiments, practice of the present invention can preferentially reduce weight gain in certain tissues and organs, e.g., in some embodiments, a phospholipase A2 inhibitor can decrease weight gain in white fat of a subject on a Western diet.

Without being limited to any particular hypothesis, these benefits may result from a number of effects brought about by reduced PLA2 activity. For example, inhibition of PLA2 activity may reduce transport of phospholipids through the gastrointestinal lumen, for example, through the small intestine apical membrane, causing a depletion of the pool of phospholipids (e.g. phosphatidylcholine) in enterocytes, particularly in mammals fed with a high fat diet. In such cases, the de novo synthesis of phospholipids may not be sufficient to sustain the high turnover of phospholipids, e.g. phosphatidylcholine, needed to carry triglycerides, for example by transport in chylomicrons (See Tso, in Fat Absorption, 1986, chapt.6 177-195, Kuksis A., Ed.), incorporated herein by reference.

PLA2 inhibition can also reduce production of 1-acyl lysophospholipids, such as 1 -acyl lysophosphatydylcholine, that may act as a signaling molecule in subsequent up-regulation pathways of fat absorption, including, for example the release of additional digestive enzymes or hormones, e.g., secretin. See, Huggins, Protection against diet-induced obesity and obesity-related insulin resistance in Group 1B-PLA2-deficient mice, Am. J. Physiol. Endocrinol. Metab. 283:E994-E1001 (2002), incorporated herein by reference.

Another aspect of the present invention provides composition, kits and methods for reducing or delaying the onset of diet-induced diabetes through weight gain. An unchecked high fat diet can produce not only weight gain, but also can contribute to diabetic insulin resistance. This resistance may be recognized by decreased insulin and leptin levels in a subject. The phospholipase inhibitors, compositions, kits and methods disclosed herein can be used in the prophylactic treatment of diet-induced diabetes, or other insulin-related conditions, e.g. in decreasing insulin and/or leptin levels in a subject on a Western diet.

In some embodiments, a lumen-localized phospholipase inhibitor is used that inhibits phospholipase A2 but does not inhibitor or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase B.

Treatment of Cholesterol-Related Conditions

The term “cholesterol-related conditions” as used herein refers to a condition in which modulating the activity of HMG-CoA reductase is desirable and/or modulating the production and/or effects of one or more products of HMG-CoA reductase is desirable. In preferred embodiments, a phospholipase inhibitor of the present invention reduces the activity of HMG-CoA reductase and/or reduces the production and/or effects of one or more products of HMG-CoA reductase. For example, a cholesterol-related condition may involve elevated levels of cholesterol, in particular, non-HDL cholesterol in plasma (e.g., elevated levels of LDL cholesterol and/or VLDL/LDL levels). Typically, a patient is considered to have high or elevated cholesterol levels based on a number of criteria, for example, see Pearlman B L, The New Cholesterol Guidelines, Postgrad Med, 2002; 112(2):13-26, incorporated herein by reference. Guidelines include serum lipid profiles, such as LDL compared with HDL levels.

Examples of cholesterol-related conditions include hypercholesterolemia, lipid disorders such as hyperlipidemia, and atherogenesis and its sequelae of cardiovascular diseases, including atherosclerosis, other vascular inflammatory conditions, myocardial infarction, ischemic stroke, occlusive stroke, and peripheral vascular diseases, as well as other conditions in which decreasing cholesterol can produce a benefit. Other cholesterol-related conditions treatable with compositions, kits, and methods of the present invention include those currently treated with statins, as well as other conditions in which decreasing cholesterol absorption can produce a benefit.

In certain embodiments, a lumen-localized phospholipase inhibitor of the present invention can be used to reduce cholesterol levels, in particular non-HDL plasma cholesterol levels, e.g. by reducing cholesterol absorption. In some preferred embodiments, the composition inhibits phospholipase A2 and at least one other gastrointestinal phospholipase in addition to phospholipase A2, such as preferably phospholipase B, and also such as phospholipase A1, phospholipase C, and/or phospholipase D.

In other embodiments of the invention, the differential activities of phospholipases can be used to treat certain phospholipase-related conditions without untoward side effects resulting from inhibiting other phospholipases. For example, in certain embodiments, a phospholipase inhibitor that inhibits PLA2, but not inhibiting or not significantly inhibiting or essentially not inhibiting, for example, PLA1, PLB, PLC, or PLD can be used to treat an insulin-related condition (e.g. diabetes) and/or a weight-related condition (e.g. obesity) without affecting, or without significantly affecting, or without essentially effecting, cholesterol absorption of a subject receiving phospholipase inhibiting treatment, e.g., when the subject is on a high fat diet.

The phospholipase inhibitors, methods, and kits disclosed herein can be used in the treatment of phospholipase-related conditions. In some preferred embodiments, these effects can be realized without a change in diet and/or activity on the part of the subject. For example, the activity of PLA2 in the gastrointestinal lumen may be inhibited to result in a decrease in fat absorption and/or a reduction in weight gain in a subject on a Western diet compared to if the subject was not receiving PLA2 inhibiting treatment. More preferably, this decrease and/or reduction occurs without a change, without a significant change, or essentially without a change, in energy expenditure and/or food intake on the part of the subject, and without a change, or without a significant change, or essentially without a change in the body temperature of the subject. Further, in preferred embodiments, a phospholipase inhibitor of the present invention can be used to offset certain negative consequences of high fat diets without affecting normal aspects of metabolism on non-high fat diets.

The present invention also includes kits that can be used to treat phospholipase-related conditions, preferably phospholipase A2-related conditions or phospholipase-related conditions induced by diet, including, but not limited to, insulin-related conditions (e.g., diabetes, particularly diabetes type 2), weight-related conditions (e.g., obesity) and/or cholesterol-related conditions. These kits comprise at least one composition of the present invention and instructions teaching the use of the kit according to the various methods described herein.

Inhibitor Formulations, Routes of Administration, and Effective Doses

The phospholipase inhibitors useful in the present invention, or pharmaceutically acceptable salts thereof, can be delivered to a patient using a number of routes or modes of administration. The term “pharmaceutically acceptable salt” means those salts which retain the biological effectiveness and properties of the compounds used in the present invention, and which are not biologically or otherwise undesirable. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxyl group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

If necessary or desirable, the phospholipase inhibitor may be administered in combination with one or more other therapeutic agents. The choice of therapeutic agent that can be co-administered with a composition of the invention will depend, in part, on the condition being treated. For example, for treating obesity, or other weight-related conditions, a phospholipase inhibitor of some embodiments of the present invention can be used in combination with a statin, a fibrate, a bile acid binder, an ezitimibe (e.g., Zetia, etc), a saponin, a lipase inhibitor (e.g. Orlistat, etc), and/or an appetite suppressant, and the like. With respect to treating insulin-related conditions, e.g., diabetes, a phospholipase inhibitor of some embodiments the present invention can be used in combination with a biguanide (e.g., Metformin), thiazolidinedione, and/or α-glucosidase inhibitor, and the like.

The phospholipase inhibitors (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries which facilitate processing of the active compounds into preparations which can be used pharmaceutically. Proper formulation is dependent upon the route of administration chosen.

The phospholipase inhibitors can be administered by direct placement, orally, and/or rectally. Preferably, the phospholipase inhibitor or the pharmaceutical composition comprising the phospholipase inhibitor is administered orally. The oral form in which the phospholipase inhibitor is administered can include a powder, tablet, capsule, solution, or emulsion. The effective amount can be administered in a single dose or in a series of doses separated by appropriate time intervals, such as hours.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions, wafers, and the like, for oral ingestion by a patient to be treated. In some embodiments, the inhibitor may be formulated as a sustained release preparation. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, mehtyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. In some embodiments, the oral formulation does not have an enteric coating.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

Suitable carriers used in formulating liquid dosage forms for oral as well as parenteral administration include non-aqueous, pharmaceutically-acceptable polar solvents such as hydrocarbons, alcohols, amides, oils, esters, ethers, ketones, and/or mixtures thereof, as well as water, saline solutions, electrolyte solutions, dextrose solutions (e.g., DW5), and/or any other aqueous, pharmaceutically acceptable liquid.

Suitable nonaqueous, pharmaceutically-acceptable polar solvents include, but are not limited to, alcohols (e.g., aliphatic or aromatic alcohols having 2-30 carbon atoms such as methanol, ethanol, propanol, isopropanol, butanol, t-butanol, hexanol, octanol, benzyl alcohol, amylene hydrate, glycerin (glycerol), glycol, hexylene glycol, lauryl alcohol, cetyl alcohol, stearyl alcohol, tetrahydrofurfuryl alcohol, fatty acid esters of fatty alcohols such as polyalkylene glycols (e.g., polyethylene glycol and/or polypropylene glycol), sorbitan, cholesterol, sucrose and the like); amides (e.g., dimethylacetamide (DMA), benzyl benzoate DMA, N,N-dimethylacetamide amides, 2-pyrrolidinone, polyvinylpyrrolidone, 1-methyl-2-pyrrolidinone, and the like); esters (e.g., 2-pyrrolidinone, 1-methyl-2-pyrrolidinone, acetate esters (such as monoacetin, diacetin, and triacetin and the like), and the like, aliphatic or aromatic esters (such as dimethylsulfoxide (DMSO), alkyl oleate, ethyl caprylate, ethyl benzoate, ethyl acetate, octanoate, benzyl benzoate, benzyl acetate, esters of glycerin such as mono, di, or tri-glyceryl citrates or tartrates, ethyl carbonate, ethyl oleate, ethyl lactate, N-methyl pyrrolidinone, fatty acid esters such as isopropyl myristrate, fatty acid esters of sorbitan, glyceryl monostearate, glyceride esters such as mono, di, or tri-glycerides, fatty acid derived PEG esters such as PEG-hydroxystearate, PEG-hydroxyoleate, and the like, pluronic 60, polyoxyethylene sorbitol oleic polyesters, polyoxyethylene sorbitan esters such as polyoxyethylene-sorbitan monooleate, polyoxyethylene-sorbitan monostearate, polyoxyethylene-sorbitan monolaurate, polyoxyethylene-sorbitan monopalmitate, alkyleneoxy modified fatty acid esters such as polyoxyl 40 hydrogenated castor oil and polyoxyethylated castor oils, saccharide fatty acid esters (i.e., the condensation product of a monosaccharide, disaccharide, or oligosaccharide or mixture thereof with a fatty acid(s)(e.g., saturated fatty acids such as caprylic acid, myristic acid, palmitic acid, capric acid, lauric acid, and stearic acid, and unsaturated fatty acids such as palmitoleic acid, oleic acid, elaidic acid, erucic acid and linoleic acid)), or steroidal esters and the like); alkyl, aryl, or cyclic ethers (e.g., diethyl ether, tetrahydrofuran, diethylene glycol monoethyl ether, dimethyl isosorbide and the like); glycofurol (tetrahydrofurfuryl alcohol polyethylene glycol ether); ketones (e.g., acetone, methyl isobutyl ketone, methyl ethyl ketone and the like); aliphatic, cycloaliphatic or aromatic hydrocarbons (e.g., benzene, cyclohexane, dichloromethane, dioxolanes, hexane, n-hexane, n-decane, n-dodecane, sulfolane, tetramethylenesulfoxide, tetramethylenesulfon, toluene, tetramethylenesulfoxide dimethylsulfoxide (DMSO) and the like); oils of mineral, animal, vegetable, essential or synthetic origin (e.g., mineral oils such as refined paraffin oil, aliphatic or wax-based hydrocarbons, aromatic hydrocarbons, mixed aliphatic and aromatic based hydrocarbons, and the like, vegetable oils such as linseed, soybean, castor, rapeseed, coconut, tung, safflower, cottonseed, groundnut, palm, olive, corn, corn germ, sesame, persic, peanut oil, and the like, as well as glycerides such as mono-, di- or triglycerides, animal oils such as cod-liver, haliver, fish, marine, sperm, squalene, squalane, polyoxyethylated castor oil, shark liver oil, oleic oils, and the like); alkyl or aryl halides e.g., methylene chloride; monoethanolamine; trolamine; petroleum benzin; omega-3 polyunsaturated fatty acids (e.g., α-linolenic acid, docosapentaenoic acid, docosahexaenoic acid, eicosapentaenoic acid, and the like); polyglycol ester of 12-hydroxystearic acid; polyethylene glycol; polyoxyethylene glycerol, and the like.

Other pharmaceutically acceptable solvents that can be used in formulating pharmaceutical compositions of a phospholipase inhibitor of the present invention including, for example, for direct placement, are well known to those of ordinary skill in the art, e.g. see Modern Pharmaceutics, (G. Banker et al., eds., 3d ed.)(Marcel Dekker, Inc., New York, N.Y., 1995), The Handbook of Pharmaceutical Excipients, (American Pharmaceutical Association, Washington, D.C.; The Pharmacological Basis of Therapeutics, (Goodman & Gilman, McGraw Hill Publishing), Remington's Pharmaceutical Sciences (A. Gennaro, ed., 19th ed.)(Mack Publishing, Easton, Pa., 1995), Pharmaceutical Dosage Forms, (H. Lieberman et al., eds.,)(Marcel Dekker, Inc., New York, N.Y., 1980); and The United States Pharmacopeia 24, The National Formulary 19, (National Publishing, Philadelphia, Pa., 2000).

Formulations for rectal administration may be prepared in the form of a suppository, an ointment, an enema, a tablet, or a cream for release of the phospholipase inhibitor in the gastrointestinal tract, e.g., the small intestine. Rectal suppositories can be made by mixing one or more phospholipase inhibitors of the present invention, or pharmaceutically acceptable salts thereof, with acceptable vehicles, for example, cocoa butter, with or without the addition of waxes to alter melting point. Acceptable vehicles can also include glycerin, salicylate and/or polyethylene glycol, which is solid at normal storage temperature, and a liquid at those temperatures suitable to release the phospholipase inhibitor inside the body, such as in the rectum. Oils may also be used in rectal formulations of the soft gelatin type and in suppositories. Water soluble suppository bases, such as polyethylene glycols of various molecular weights, may also be used. Suspension formulations may be prepared that use water, saline, aqueous dextrose and related sugar solutions, and glycerols, as well as suspending agents such as pectins, carbomers, methyl cellulose, hydroxypropyl cellulose or carboxymethyl cellulose, as well as buffers and preservatives.

Pharmaceutical compositions suitable for use in the present invention include compositions wherein the active ingredients are present in an effective amount, i.e., in an amount sufficient to produce a therapeutic and/or a prophylactic benefit in at least one condition being treated. The actual amount effective for a particular application will depend on the condition being treated and the route of administration. Determination of an effective amount is well within the capabilities of those skilled in the art, especially in light of the disclosure herein. For example, the IC50 values and ranges provided in Table 1 above provide guidance to enable one of ordinary skill in the art to select effective dosages of the corresponding phospholipase inhibiting moieties.

The effective amount when referring to a phospholipase inhibitor will generally mean the dose ranges, modes of administration, formulations, etc., that have been recommended or approved by any of the various regulatory or advisory organizations in the medical or pharmaceutical arts (eg, FDA, AMA) or by the manufacturer or supplier. Effective amounts of phospholipase inhibitors can be found, for example, in the Physicians Desk Reference. The effective amount when referring to producing a benefit in treating a phospholipase-related condition, such as insulin-related conditions (e.g., diabetes), weight-related conditions (e.g., obesity), and/or cholesterol related-conditions will generally mean the levels that achieve clinical results recommended or approved by any of the various regulatory or advisory organizations in the medical or pharmaceutical arts (eg, FDA, AMA) or by the manufacturer or supplier.

A person of ordinary skill using techniques known in the art can determine the effective amount of the phospholipase inhibitor. In the present invention, the effective amount of a phospholipase inhibitor localized in the gastsrointestinal lumen can be less than the amount administered in the absence of such localization. Even a small decrease in the amount of phospholipase inhibitor administered is considered useful for the present invention. A significant decrease or a statistically significant decrease in the effective amount of the phospholipase inhibitor is particularly preferred. In some embodiments of the invention, the phospholipase inhibitor reduces activity of phospholipase to a greater extent compared to non-lumen localized inhibitors. Lumen-localization of the phospholipase inhibitor can decrease the effective amount necessary for the treatment of phospholipase-related conditions, such as insulin-related conditions (e.g., diabetes), weight-related conditions (e.g., obesity) and/or cholesterol-related conditions by about 5% to about 95%. The amount of phospholipase inhibitor used could be the same as the recommended dosage or higher than this dose or lower than the recommended dose.

In some embodiments, the recommended dosage of a phospholipase inhibitor is between about 0.1 mg/kg/day and about 1,000 mg/kg/day. The effective amount for use in humans can be determined from animal models. For example, a dose for humans can be formulated to achieve circulating and/or gastrointestinal concentrations that have been found to be effective in animals, e.g. a mouse model as the ones described in the samples below.

A person of ordinary skill in the art can determine phospholipase inhibition by measuring the amount of a product of a phospholipase, e.g., lysophosphatidylcholine (LPC), a product of PLA2. The amount of LPC can be determined, for example, by measuring small intestine, lymphatic, and/or serum levels post-prandially. Another technique for determining amount of phospholipase inhibition involves taking direct fluid samples from the gastrointestinal tract. A person of ordinary skill in the art would also be able to monitor in a patient the effect of a phospholipase inhibitor of the present invention, e.g., by monitoring cholesterol and/or triglyceride serum levels. Other techniques would be apparent to one of ordinary skill in the art. Other approaches for measuring phospholipase inhibition and/or for demonstrating the effects of phospholipase inhibitors of some embodiments are further illustrated in the examples below.

### EXAMPLES

### Example 1

### Reduction in Insulin Resistance in a Mouse Model

A phospholipase inhibitor, for example a composition comprising a phospholipase inhibiting moiety disclosed herein, can be used in a mouse model to demonstrate, for example, suppression of diet-induced insulin resistance, relating to, for example, diet-induced onset of diabetes. The phospholipase inhibitor can be administered to subject animals either as a chow supplement and/or by oral gavage BID in a certain dosage (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

The duration of the study is typically about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight.

At the end of the about 6 to about 8 week study period, an oral glucose tolerance test and/or an insulin sensitivity test can be conducted as follows:

Oral glucose tolerance test—after an overnight fast, mice from each dosing group can be fed a glucose bolus (e.g., by stomach gavage using about 2 g/kg body weight) in about 50 μl of saline. Blood samples can be obtained from the tail vein before, and about 15, about 30, about 60, and about 120 minutes after glucose administration; blood glucose levels at the various time points can then be determined.

Insulin sensitivity test—after about a 6 hour morning fast, mice in each of the dosing groups can be administered bovine insulin (e.g., about 1 U/kg body weight, using, e.g., intraperitoneal administration. Blood samples can be obtained from the tail vein before, and about 15, about 30, about 60, and about 120 minutes after insulin administration; plasma insulin levels at the various time points can then be determined, e.g., by radioimmunoassay.

The effect of the non-absorbed phospholipase inhibitor, e.g., a phospholipase A2 inhibitor, is a decrease in insulin resistance, i.e., better tolerance to glucose challenge by, for example, increasing the efficiency of glucose metabolism in cells, and in the animals of the dose-treated groups fed a Western (high fat/high cholesterol) diet relative to the animals of the control groups. Effective dosages can also be determined.

### Example 2

### Reduction in Fat Absorption in a Mouse Model

A phospholipase inhibitor, for example a composition comprising a phospholipase inhibiting moiety disclosed herein, can be used in a mouse model to demonstrate, for example, reduced lipid absorption in subjects on a Western diet. The phospholipase inhibitor can be administered to subject animals either as a chow supplement and/or by oral gavage BID in a certain dosage (e.g., less than about 1 ml/kg body weight, or about 25 to about 50 μL/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to 90 mg/kg body weight.

The mouse chow used may have a composition representative of a Western-type (high fat and/or high cholesterol) diet. For example, the chow may contain about 21% milk fat and about 0. 15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Triglyceride measurements can be taken for a duration of about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight. On a weekly basis, plasma samples can be obtained from the subject animals and analyzed for total triglycerides, for example, to determine the amount of lipids absorbed into the blood circulation.

The effect of the non-absorbed phospholipase inhibitor, e.g., a phospholipase A2 inhibitor, is a net decrease in lipid plasma levels, which indicates reduced fat absorption, in the animals of the dose-treated groups fed a Western (high fat/high cholesterol) diet relative to the animals of the control groups. Effective dosages can also be determined.

### Example 3

### Reduction in Diet-Induced Hypercholesterolemia in a Mouse Model

A phospholipase inhibitor, for example a composition comprising a phospholipase inhibiting moiety disclosed herein, can be used in a mouse model to demonstrate, for example, suppression of diet-induced hypercholesterolemia. The phospholipase inhibitor can be administered to subject animals either as a chow supplement and/or by oral gavage BID (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western-type (high fat and/or high cholesterol) diet. For example, the chow may contain about 21% milk fat and about 0. 15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Cholesterol and/or triglyceride measurements can be taken for a duration of about 6 to about 8 weeks, with the subject animals being dosed every day during this period. Typical dosing groups, containing about 6 to about 8 animals per group, can be composed of a untreated control group, a vehicle control group, and dose-treated groups ranging from about 10 mg/kg body weight to about 90 mg/kg body weight. On a weekly basis, plasma samples can be obtained from the subject animals and analyzed for total cholesterol and/or triglycerides, for example, to determine the amount of cholesterol and/or lipids absorbed into the blood circulation. Since most plasma cholesterol in a mouse is associated with HDL (in contrast to the LDL association of most cholesterol in humans), HDL and non-HDL fractions can be separated to aid determination of the effectiveness of the non-absorbed phospholipase inhibitor in lowering plasma non-HDL levels, for example VLDL/LDL.

The effect of the non-absorbed phospholipase inhibitor, e.g., a phospholipase A2 inhibitor, is a net decrease in hypercholesterolemia in the animals of the dose-treated groups fed a Western (high fat/high cholesterol) diet relative to the animals of the control groups. Effective dosages can also be determined.

All publications, patents, and patent applications mentioned in this specification are herein incorporated by reference to the same extent as if each individual publication, patent, or patent application was specifically and individually indicated to be incorporated by reference.

It can be appreciated to one of ordinary skill in the art that many changes and modifications can be made thereto without departing from the spirit or scope of the appended claims, and such changes and modifications are contemplated within the scope of the instant invention.

1. A composition comprising a phospholipase inhibitor wherein said inhibitor is localized in a gastrointestinal lumen

2. The composition as recited in claim 1 wherein said inhibitor is not absorbed through a gastrointestinal mucosa.

3. The composition as recited in claim 1 wherein said inhibitor essentially does not inhibit a lipase.

4. The composition as recited in claim 1 wherein said inhibitor inhibits phospholipase A2.

5. The composition as recited in claim I wherein said inhibitor inhibits phospholipase A2 and phospholipase B.

6. The composition as recited in claim 1 wherein said inhibitor inhibits phospholipase A2 and essentially does not inhibit phospholipase B.

7. The composition as recited in claim 1 wherein said inhibitor does not act on said gastrointestinal mucosa.

8. The composition as recited in claim 1 wherein said inhibitor has a permeability coefficient lower than about −5.

9. The composition as recited in claim 1 wherein said inhibitor comprises at least one moiety selected from an oligomer, a polymer, a hydrophobic moiety, a hydrophilic moiety, and a charged moiety.

10. The composition as recited in claim 1 wherein said inhibitor comprises a repeat unit of formula 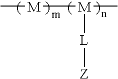
wherein n is an integer, m is an integer, M is a polymer moiety, L is a linking moiety and Z is a phospholipase inhibiting moiety.

11. The composition as recited in claim 10 wherein said phospholipase inhibiting moiety Z is a phospholipase A2 inhibiting moiety.

12. The composition as recited in claim 11 wherein n is less than about 500.

13. The composition as recited in claim 11 wherein said phospholipase A2 inhibiting moiety Z is a small molecule.

14. The composition as recited in claim 11 wherein said phospholipase A2 inhibiting moiety is at least one compound selected from an arachidonic acid analogue; an arachidonyl trifluoromethyl ketone; a methylarachidonyl fluorophosphonate; a palmitoyl trifluoromethyl ketone; a benzensulfonamide derivative, a bromoenol lactone, a p-bromophenyl bromide, a bromophenacyl bromide, a trifluoromethylketone, a sialoglycolipid and a proteoglycan.

15. The composition as recited in claim 11 wherein said phospholipase A2 inhibiting moiety is a phospholipid analog or a transition state analog.

16. The composition as recited in claim 15 wherein said phospholipid analog or said transition state analog is linked via a hydrophobic group of said phospholipid analog or of said transition state analog.

17. The composition as recited in claim 15 wherein said phospholipid analog or said transition state analog is at least one structure selected from 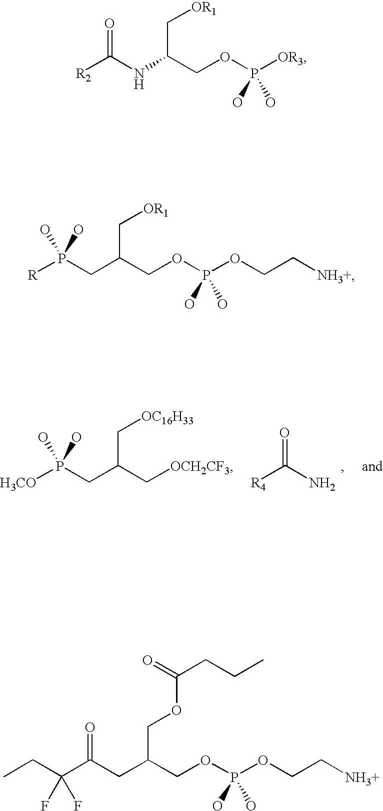
wherein R is alkyl or O-alkyl; R₁ is alkyl or C(═O)alkyl; R₂ is alkyl; R₃ is —(CH₂)ₙ—NH₃⁺, (CH₂)ₙ—OH or —(CH₂)ₙ—N(R′)₃⁺ where n=2-4 and R′ is hydrogen or alkyl; and R₄ is oleyl, elaidoyl, petroselaidoyl, gamma-lineoyl, or arachidonyl.

18. The composition as recited in claim 15 wherein said phospholipid analog or said transition state analog is at least one structure selected from

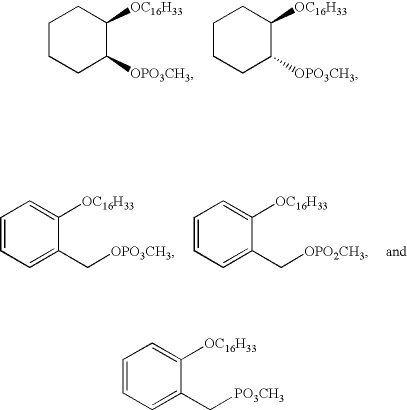

19. The composition as recited in claim 11 wherein said phospholipase A2 inhibiting moiety Z is at least one structure selected from

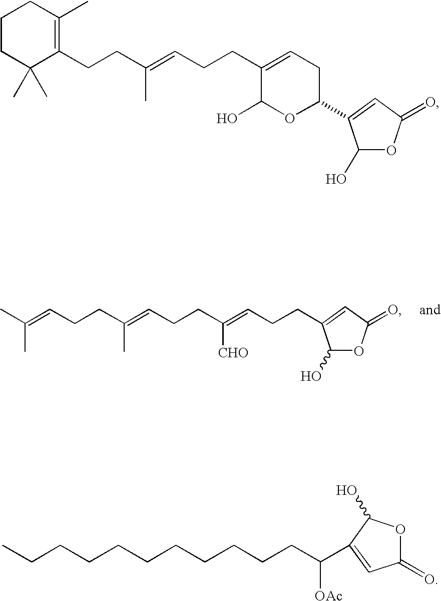

20. The composition as recited in claim 11 wherein said wherein phospholioase A2 inhibiting moiety Z is at least one compound selected from 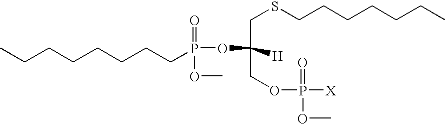
wherein X is 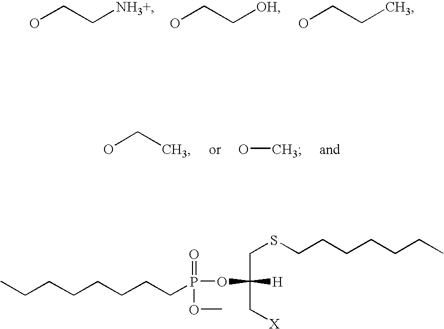
wherein X is OH,

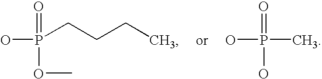

21. The composition as recited in claim 11 wherein said wherein phospholioase A2 inhibiting moiety Z is

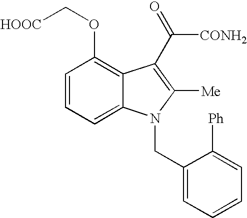

22. The composition as recited in claim 11 wherein said polymer moiety M is at least one selected from an aliphatic, an alicyclic, and an aromatic polymer moiety.

23. The composition as recited in claim 11 wherein said polymer moiety M is at least one selected from a carboxymethylcellulose, a chitosan, and a sulfoethylcellulose polymer moiety.

24. The composition as recited in claim 11 wherein said polymer moiety M comprises at least one monomer selected from an acrylic, a methacrylic, a vinylic, an allylic and a styrenic monomer.

25. The composition as recited in claim 1 wherein said inhibitor is localized in said gastrointestinal lumen as a result of efflux from a gastrointestinal mucosal cell.

26. The composition as recited in claim 25 wherein said inhibitor comprises a recognition motif moiety.

27. The composition as recited in claim 26 wherein said inhibitor further comprises an efflux enhancing moiety.

28. The composition as recited in claim 1 wherein said inhibitor hinders access of a phospholipase to a phospholipid substrate.

29. The composition as recited in claim 28 wherein said inhibitor interacts with a lipid-water interface.

30. The composition as recited in claim 28 wherein said inhibitor interacts with a phospholipase.

31. The composition as recited in claim 28 wherein said inhibitor comprises a polymer moiety.

32. The composition as recited in claim 31 wherein said polymer moiety interacts with the i-face of a phospholipase.

33. The composition as recited in claim 28 wherein said inhibitor comprises a polymer moiety and a phospholipase inhibiting moiety.

34. The composition as recited in claim 33 wherein said polymer moiety interacts with the i-face of a phospholipase.

35. The composition as recited in claim 33 wherein said polymer moiety is dendritic.

36. The composition as recited in claim 33 wherein said inhibitor further comprises a repeat unit of formula 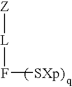
wherein Z is said phospholipase inhibiting moiety, L is a linking moiety; F is focal point where covalent linkages from a plurality of segments SXp converge; S is a spacer moiety; X is an anionic moiety, p is 1, 2, 3, or 4, and q is 2, 3, 4, 5, 6, 7, or 8.

37. The composition as recited in claim 36 wherein said phospholipase inhibiting moiety Z is a phospholipase A2 inhibiting moiety.

38. The composition as recited in claim 36 wherein said anionic moiety X is at least one acidic group selected from a carboxylate group, a sulfonate group, a sulfate group, a sulfamate group, a phosphoramidate group, a phosphate group, a phosphonategroup, a phosphinate group, and a gluconate group.

39. The composition as recited in claim 36 wherein said anionic moiety is arranged so as to bind to a cationic ring of the i-face of PLA2.

40. The composition as recited in claim 36 wherein said spacer moiety is arranged so as to bind to a hydrophobic crown of the i-face of PLA2.

41. The composition as recited in claim 1 wherein said inhibitor reversibly inhibits phospholipase A2.

42. The composition as recited in claim 41 wherein said inhibitor reversibly inhibits phospholipase A2 by at least one mechanism selected from noncompetitive and uncompetitive inhibition.

43. The composition as recited in claim 41 wherein said inhibitor reversibly inhibits phospholipase A2 by uncompetitive inhibition.

44. The composition as recited in claim 41 wherein said inhibitor binds phospholipase A2 to effect an allosteric change

45. The composition as recited in claim 1 wherein said inhibitor irreversibly inhibits phospholipase A2.

46. The composition as recited in claim 45 wherein said inhibitor reduces re-absorption of secreted phospholipase A2 through a gastrointestinal mucosa.

47. The composition as recited in claim 1 wherein said inhibitor produces a therapeutic and/or prophylatic benefit in treating an insulin-related condition in a subject receiving said inhibitor.

48. The composition as recited in claim 1 wherein said inhibitor produces a therapeutic and/or prophylactic benefit in treating diabetes type 2 in a subject receiving said inhibitor.

49. The composition as recited in claim 1 wherein said inhibitor increases insulin sensitivity in a subject receiving said inhibitor.

50. The composition as recited in claim 1 wherein said inhibitor improves glucose tolerance in a subject receiving said inhibitor.

51. The composition as recited in claim 1 wherein said inhibitor decreases fasting blood insulin levels in a subject receiving said inhibitor

52. The composition as recited in claim 1 wherein said inhibitor produces a therapeutic and/or prophylactic benefit in treating a weight-related condition in a subject receiving said inhibitor.

53. The composition as recited in claim 1 wherein said inhibitor decreases fat absorption in a subject on a Western diet receiving said inhibitor.

54. The composition as recited in claim 1 wherein said inhibitor increases lipid excretion from a subject on a Western diet receiving said inhibitor.

55. The composition as recited in claim 1 wherein said inhibitor decreases obesity in a subject on a Western diet receiving said inhibitor.

56. The composition as recited in claim 1 wherein said inhibitor decreases weight gain in a subject on a Western diet receiving said inhibitor.

57. The composition as recited in claim 1 wherein said inhibitor decreases insulin levels in a subject on a Western diet receiving said inhibitor.

58. The composition as recited in claim 1 wherein said inhibitor decreases leptin levels in a subject on a Western diet receiving said inhibitor.

59. The composition as recited in claim 1 wherein said inhibitor reduces on delays or prevents onset of diet-induced diabetes in a subject receiving said inhibitor.

60. The composition as recited in claim 1 wherein said inhibitor produces a therapeutic and/or prophylactic benefit in treating a cholesterol-related condition and an insulin-related condition in a subject receiving said inhibitor.

61. The composition as recited in claim 60 wherein said cholesterol-related condition is hypercholesterolemia.

62. The composition as recited in claim 60 wherein said insulin-related condition is diabetes type 2.

63. The composition as recited in claim 1 wherein said inhibitor produces a decrease in cholesterol absorption in a subject receiving said inhibitor.

64. The composition as recited in claim 1 wherein said inhibitor produces a therapeutic and/or prophylactic benefit in treating a cholesterol-related condition in a subject receiving said inhibitor.

65. The composition as recited in claim 64 wherein said cholesterol-related condition is hypercholesterolemia.

66.-89. (canceled)
